(19) United States
(12) Patent Application Publication
(10) Pub. No.: US 2010/0113550 A1
(43) Pub. Date: May 6, 2010

(54) New Crystal Forms

(75) Inventors: Alexander Beliaev, Mindelo, Vila do Conde (PT); David Learmonth, Alfena (PT); Valeriya Smolenskaya, Lafayette, IN (US)

Correspondence Address: CONLEY ROSE, P.C., 5601 GRANITE PARKWAY, SUITE 750, PLANO, TX, 75024, US

(73) Assignee: BIAL - PORTELA AND CA, S.A., S. Mamede do Coronado (PT)

(21) Appl. No.: 12/301,692

(22) PCT Filed: May 31, 2007
(86) PCT No.: PCT/PT07/00023

§ 371 Date: Jan. 23, 2009

(30) Foreign Application Priority Data
May 31, 2006 (GB) 0610804.7
Apr. 4, 2007 (GB) 0706647.5

(51) Int. Cl. A61K 31/4178; C07D 233/42; A61P 9/00

(52) U.S. Cl. 514/397; 548/311.4

(57) ABSTRACT

Polymorphs of (R)-5-(2-Aminoethyl)-1-(6,8-difluorochroman-3-yl)-1,3-dihydroimidazole-2-thione hydrochloride and methods of their preparation.

This invention relates to polymorphs of (R)-5-(2-Aminoethyl)-1-(6,8-difluorochroman-3-yl)-1,3-dihydroimidazole-2-thione hydrochloride and methods of their preparation.

(R)-5-(2-Aminoethyl)-1-(6,8-difluorochroman-3-yl)-1,3-dihydroimidazole-2-thione hydrochloride (the compound of formula 1, below) is a potent, non-toxic and peripherally selective inhibitor of D b H, which can be used for treatment of certain cardiovascular disorders. It is disclosed in WO2004/033447, along with processes for its preparation.

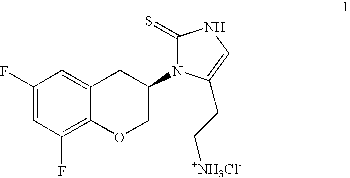

The process disclosed in WO2004/033447 for preparing compound 1 (see example 16) results in the amorphous form of compound 1. The process of example 16 is described in WO2004/033447 on page 5, lines 16 to 21 and in Scheme 2 on page 7. Prior to formation of compound 1, a mixture of intermediates is formed (compounds V and VI in scheme 2). The mixture of intermediates is subjected to a high concentration of HCl in ethyl acetate. Under these conditions, the primary product of the reaction is compound 1, which precipitates as it forms as the amorphous form.

The present invention provides crystalline polymorphs of compound 1 which exhibit higher purity than the amorphous form prepared by the WO2004/033447 process. The crystalline forms are prepared from crystallisation or recrystallisation of pre-formed compound 1 (either the amorphous faun or one of the other crystalline forms). The present invention also provides a characterisation of the amorphous form of compound 1 and processes for its preparation. The amorphous form produced according to the processes of the present invention is also a part of the present invention.

The present invention further provides improved processes for preparing compound 1. The processes can be used to produce the precursor compound 1 in the preparation of the polymorphs and amorphous form of compound 1 of the present invention.

According to a first aspect of the present invention, there is provided crystalline Form A of (R)-5-(2-Aminoethyl)-1-(6,8-difluorochroman-3-yl)-1,3-dihydroimidazole-2-thione hydrochloride having an XRPD pattern with peaks at 8.3 and 26.8±0.2° 2#. The XRPD pattern for crystalline Form A may have further peaks at 15.0, 16.2, and 24.2±0.2° 2#. The XRPD pattern for crystalline Form A may have still further peaks at 4.9, 12.9, 19.8, 21.8 and 22.9±0.2° 2#.

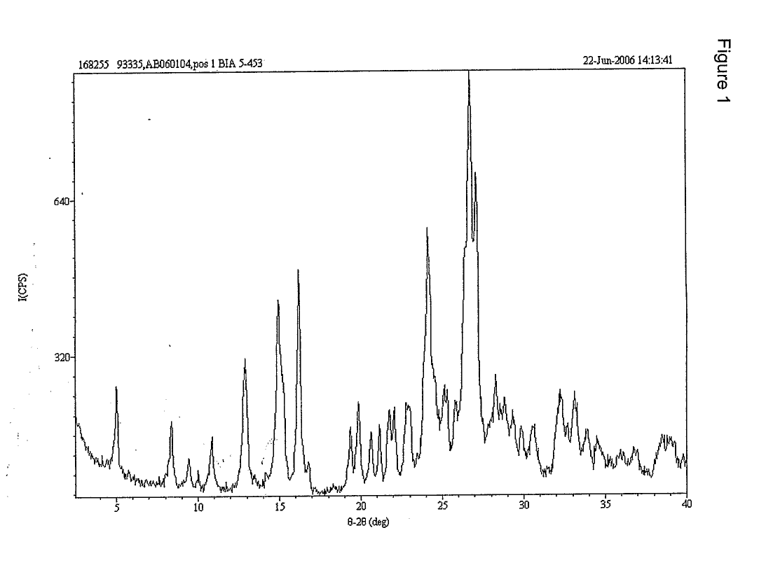

According to another aspect of the invention, there is provided crystalline Form A of (R)-5-(2-Aminoethyl)-1-(6,8-difluorochroman-3-yl)-1,3-dihydroimidazole-2-thione hydrochloride having the XRPD pattern of FIG. 1.

In an embodiment, crystalline Form A of (R)-5-(2-Aminoethyl)-1-(6,8-difluorochroman-3-yl)-1,3-dihydroimidazole-2-thione hydrochloride is a variable hydrate with the number of moles of water being dependent on the relative humidity and varying from about 0.09 to about 0.65 moles.

According to another aspect of the present invention, there is provided crystalline Form A of (R)-5-(2-Aminoethyl)-1-(6,8-difluorochroman-3-yl)-1,3-dihydroimidazole-2-thione hydrochloride having characteristic FT-IR peaks at 1491.90, 1220.70, 1117.50, 1039.50, 851.80 and 747.00 cm⁻¹. The crystalline Form A of (R)-5-(2-Aminoethyl)-1-(6,8-difluorochroman-3-yl)-1,3-dihydroimidazole-2-thione hydrochloride may have further characteristic FT-IR peaks at 3053.30, 1599.80, 1406.10, 1330.70, 1287.60, 1194.00, 985.50 and 713.70 cm⁻¹. The crystalline Form A of (R)-5-(2-Aminoethyl)-1-(6,8-difluorochroman-3-yl)-1,3-dihydroimidazole-2-thione hydrochloride may have still further characteristic FT-IR peaks at 2939.70, 1448.30 and 1244.50 cm⁻¹.

According to another aspect of the present invention, there is provided crystalline Form A of (R)-5-(2-Aminoethyl)-1-(6,8-difluorochroman-3-yl)-1,3-dihydroimidazole-2-thione hydrochloride having the FT-IR spectrum of FIG. 6.

According to another aspect of the present invention, there is provided crystalline Form A of (R)-5-(2-Aminoethyl)-1-(6,8-difluorochroman-3-yl)-1,3-dihydroimidazole-2-thione hydrochloride having the DSC thermogram of FIG. 9.

According to another aspect of the present invention, there is provided crystalline Form A of (R)-5-(2-Aminoethyl)-1-(6,8-difluorochroman-3-yl)-1,3-dihydroimidazole-2-thione hydrochloride having a purity greater than or equal to 99.0%. The purity may be in the range of 99.0% to 99.9%. In an embodiment, the purity may be in the range of 99.0% to 99.8%. In particular, the purity may be in the range of 99.2% to 99.8%. More particularly, the crystalline Form A of (R)-5-(2-Aminoethyl)-1-(6,8-difluorochroman-3-yl)-1,3-dihydroimidazole-2-thione hydrochloride may have a purity of 99.5%.

According to another aspect of the present invention, there is provided crystalline Form B of (R)-5-(2-Aminoethyl)-1-(6,8-difluorochroman-3-yl)-1,3-dihydroimidazole-2-thione hydrochloride having an XRPD pattern with peaks at 8.0 and 8.6±0.2° 2#. The XRPD pattern for crystalline Form B may have further peaks at 13.6, 14.4, 16.0, 24.3 and 26.7±0.2° 2#. The XRPD pattern for crystalline Form B may have still further peaks at 4.8, 12.7, 13.6, 14.4, 15.2, 21.7 and 22.9±0.2° 2#.

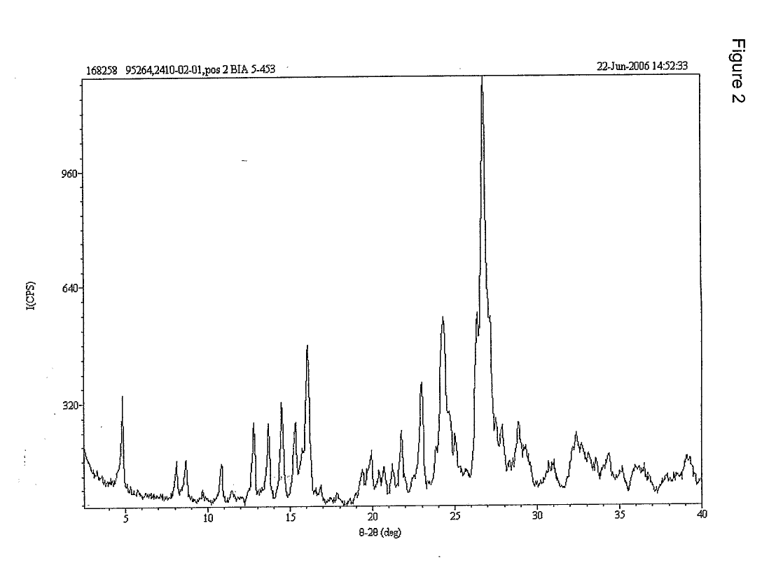

According to another aspect of the present invention, there is provided crystalline Form B of (R)-5-(2-Aminoethyl)-1-(6,8-difluorochroman-3-yl)-1,3-dihydroimidazole-2-thione hydrochloride having the XRPD pattern of FIG. 2.

In an embodiment, crystalline Form B of (R)-5-(2-Aminoethyl)-1-(6,8-difluorochroman-3-yl)-1,3-dihydroimidazole-2-thione hydrochloride is a variable hydrate with the number of moles of water being dependent on the relative humidity and varying from about 1.1 to about 1.4 moles. In a further embodiment, crystalline Form B of (R)-5-(2-Aminoethyl)-1-(6,8-difluorochroman-3-yl)-1,3-dihydroimidazole-2-thione hydrochloride is a monohydrate.

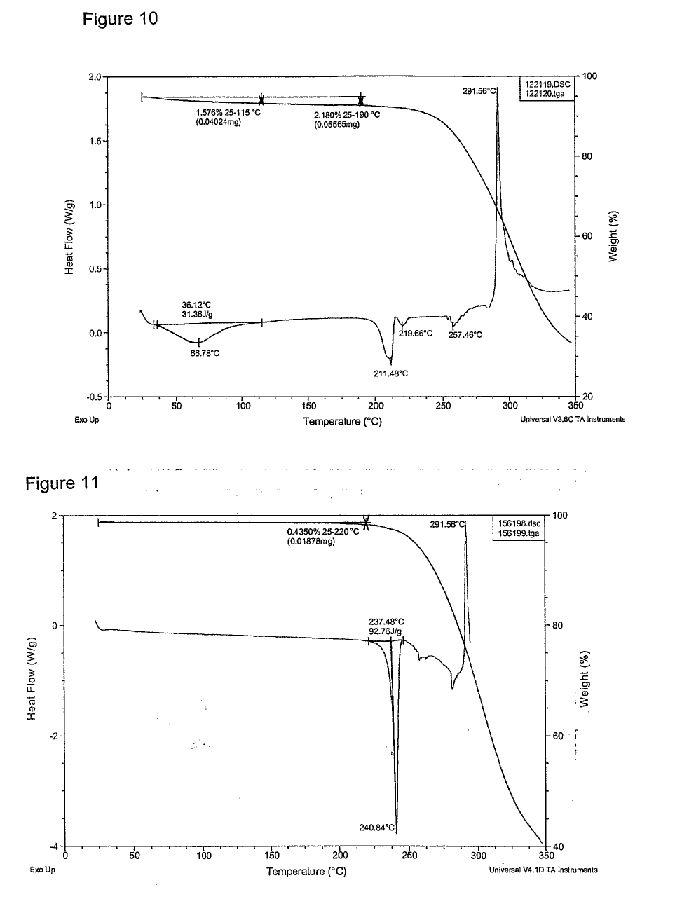

According to another aspect of the present invention, there is provided crystalline Form B of (R)-5-(2-Aminoethyl)-1-(6,8-difluorochroman-3-yl)-1,3-dihydroimidazole-2-thione hydrochloride having the DSC thermogram of FIG. 10.

According to another aspect of the present invention, there is provided crystalline Form B of (R)-5-(2-Aminoethyl)-1-(6,8-difluorochroman-3-yl)-1,3-dihydroimidazole-2-thione hydrochloride having a purity greater than or equal to 99.0%. The purity may be in the range of 99.0% to 99.9%. For example, the purity may be in the range of 99.0% to 99.8%. In particular, the purity may be in the range of 99.2% to 99.8%. More particularly, the crystalline Form B of (R)-5-(2-Aminoethyl)-1-(6,8-difluorochroman-3-yl)-1,3-dihydroimidazole-2-thione hydrochloride may have a purity of 99.5%.

According to another aspect of the present invention, there is provided crystalline Form C of (R)-5-(2-Aminoethyl)-1-(6,8-difluorochroman-3-yl)-1,3-dihydroimidazole-2-thione hydrochloride having an XRPD pattern with peaks at 13.9, 18.1, 22.1, 25.1 and 25.7±0.2° 2#. The XRPD pattern for Form C may have further peaks at 15.3, 17.7 and 20.2±0.2° 2#. The XRPD pattern for Form C may have still further peaks at 16.2, 16.7, 21.0 and 24.2 0.2° 2#.

According to another aspect of the present invention, there is provided crystalline Form C of (R)-5-(2-Aminoethyl)-1-(6,8-difluorochroman-3-yl)-1,3-dihydroimidazole-2-thione hydrochloride having the XRPD pattern of FIG. 3.

According to another aspect of the present invention, there is provided crystalline Form C of (R)-5-(2-Aminoethyl)-1-(6,8-difluorochroman-3-yl)-1,3-dihydroimidazole-2-thione hydrochloride having characteristic FT-IR peaks at 1492, 1220.2, 1117.4, 1033.4, 845.2, 792.6 and 750.1 cm⁻¹. The crystalline Form C may have further characteristic FT-IR peaks at 3041.70, 1596.50, 1403.40, 1333.80, 1290.90, 1173.20, 1078.10, 984.90 and 713.20 cm⁻¹.

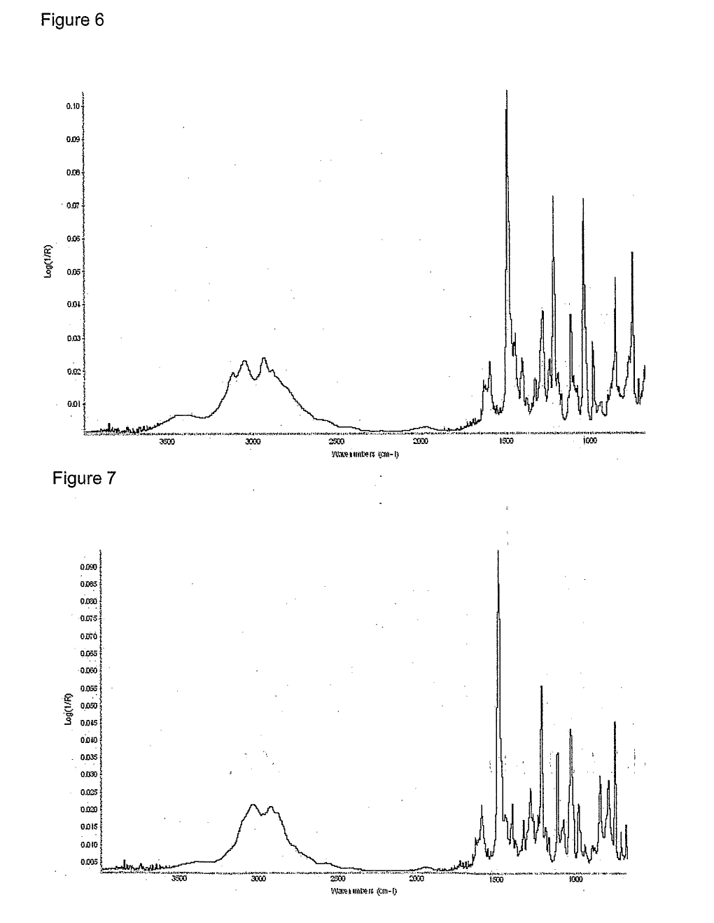

According to another aspect of the present invention, there is provided crystalline Form C of (R)-5-(2-Aminoethyl)-1-(6,8-difluorochroman-3-yl)-1,3-dihydroimidazole-2-thione hydrochloride having the FT-IR spectrum of FIG. 7.

According to another aspect of the present invention, there is provided crystalline Form C of (R)-5-(2-Aminoethyl)-1-(6,8-difluorochroman-3-yl)-1,3-dihydroimidazole-2-thione hydrochloride having a purity greater than or equal to 99.0%. The purity may be in the range of 99.0% to 99.9%. For example, the purity may be in the range of 99.0% to 99.8%. In particular, the purity may be in the range of 99.2% to 99.8%. More particularly, the crystalline Form C of (R)-5-(2-Aminoethyl)-1-(6,8-difluorochroman-3-yl)-1,3-dihydroimidazole-2-thione hydrochloride may have a purity of 99.5%.

According to another aspect of the present invention, there is provided crystalline Form X of (R)-5-(2-Aminoethyl)-1-(6,8-difluorochroman-3-yl)-1,3-dihydroimidazole-2-thione hydrochloride having an XRPD pattern with peaks at 5.4, 10.2, 12.4 and 18.6±° 2#. The XRPD pattern for crystalline Form X may have further peaks at 6.2, 9.5, 11.2 and 16.2±° 2#.

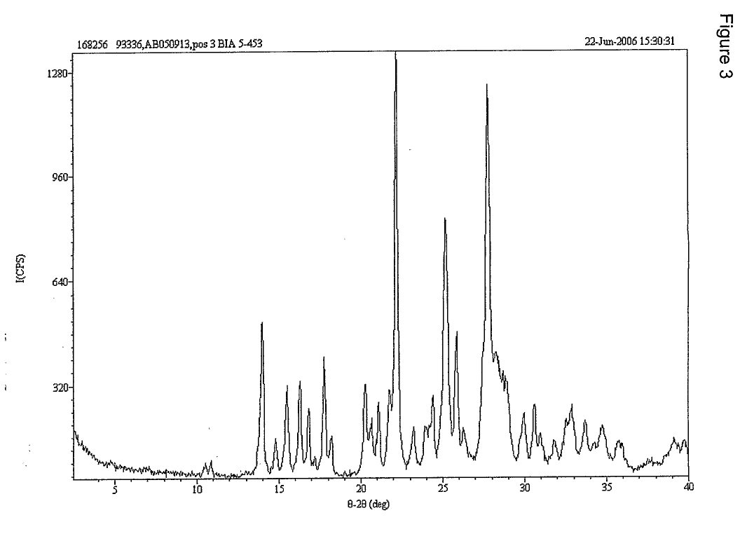

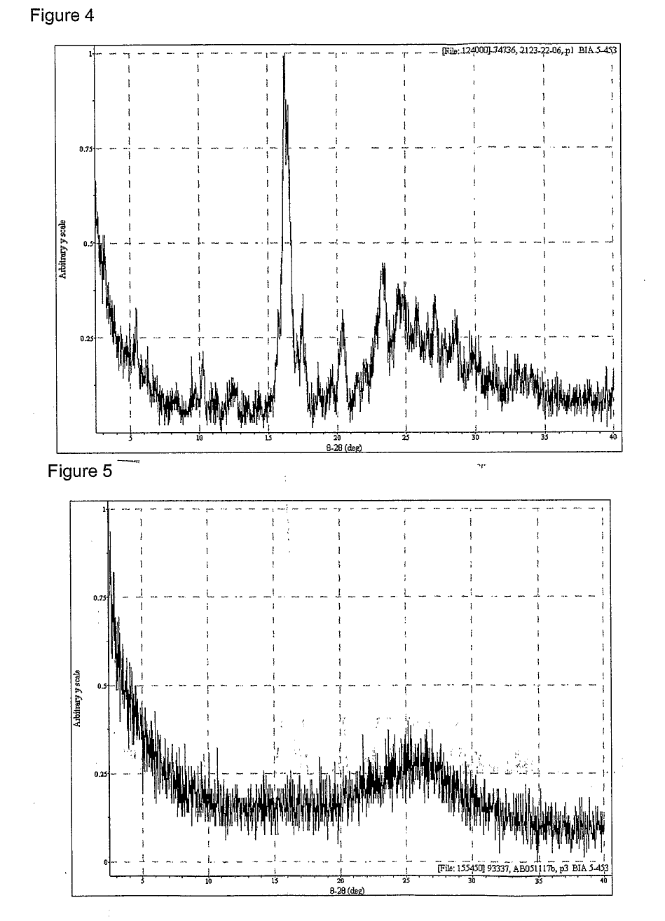

According to another aspect of the present invention, there is provided crystalline Form X of (R)-5-(2-Aminoethyl)-1-(6,8-difluorochroman-3-yl)-1,3-dihydroimidazole-2-thione hydrochloride having the XRPD pattern of FIG. 4.

According to another aspect of the present invention, there is provided crystalline Form X of (R)-5-(2-Aminoethyl)-1-(6,8-difluorochroman-3-yl)-1,3-dihydroimidazole-2-thione hydrochloride having a purity greater than or equal to 99.0%. The purity may be in the range of 99.0% to 99.9%. For example, the purity may be in the range of 99.0% to 99.8%. In particular, the purity may be in the range of 99.2% to 99.8%. More particularly, the crystalline Form X of (R)-5-(2-Aminoethyl)-1-(6,8-difluorochroman-3-yl)-1,3-dihydroimidazole-2-thione hydrochloride may have a purity of 99.5%.

According to another aspect of the present invention, there is provided a process for preparing crystalline Form A of (R)-5-(2-Amino ethyl)-1-(6,8-difluorochroman-3-yl)-1,3-dihydroimidazole-2-thione hydrochloride comprising recrystallising (R)-5-(2-Aminoethyl)-1-(6,8-difluorochroman-3-yl)-1,3-dihydroimidazole-2-thione hydrochloride in aqueous HCl.

In an embodiment, the recrystallisation comprises (a) dissolving (R)-5-(2-Amino ethyl)-1-(6,8-difluorochroman-3-yl)-1,3-dihydroimidazole-2-thione hydrochloride in aqueous HCl, (b) filtering the solution, (c) cooling the solution with stirring, and (d) isolating, washing and drying the precipitated Form A.

According to another aspect of the present invention, there is provided a process for preparing crystalline Form A of (R)-5-(2-Amino ethyl)-1-(6,8-difluorochroman-3-yl)-1,3-dihydroimidazole-2-thione hydrochloride comprising forming (R)-5-(2-Aminoethyl)-1-(6,8-difluorochroman-3-yl)-1,3-dihydroimidazole-2-thione hydrochloride in situ and crystallising Form A using aqueous HCl. Thus, Form A of compound 1 crystallises and may be isolated, followed by optional recrystallisation to form one of the polymorphic forms.

In an embodiment, the crystallisation comprises (a) adding aqueous HCl to a solution of (R)-5-(2-Aminoethyl)-1-(6,8-difluorochroman-3-yl)-1,3-dihydroimidazole-2-thione hydrochloride, (b) cooling the solution with stirring and (c) isolating, washing and drying the precipitated Form A.

According to another aspect of the present invention, there is provided a process for preparing crystalline Form B of (R)-5-(2-Aminoethyl)-1-(6,8-difluorochroman-3-yl)-1,3-dihydroimidazole-2-thione hydrochloride comprising subjecting Form A of (R)-5-(2-Aminoethyl)-1-(6,8-difluorochroman-3-yl)-1,3-dihydroimidazole-2-thione hydrochloride to 43% to 90% relative humidity.

In an embodiment, the relative humidity is from 55% to 65%.

The subjecting step may take place within a time range from 1 day to 2 weeks. In an embodiment, the subjecting step takes place over 1 to 2 days. Preferably, the subjecting step takes place at 25° C.

According to another aspect of the present invention, there is provided a process for preparing crystalline Form B of (R)-5-(2-Aminoethyl)-1-(6,8-difluorochroman-3-yl)-1,3-dihydroimidazole-2-thione hydrochloride comprising dissolving or slurrying Form A of (R)-5-(2-Aminoethyl)-1-(6,8-difluorochroman-3-yl)-1,3-dihydroimidazole-2-thione hydrochloride in an organic solvent, or mixtures of organic solvents, filtering the solution and allowing the solvent to evaporate.

The organic solvent may be selected from ethyl ether, hexane, acetonitrile, 1,4-dioxane, ethanol, ethyl acetate, hexafluoroisopropanol, methanol, methylene chloride, methyl ethyl ketone, toluene, propionitrile, trifluorotoluene, cyclohexane, methyl iso-butyl ketone, n-butyl acetate, acetone, toluene, iso-propyl ether and mixtures thereof.

In an embodiment, the solvent is allowed to evaporate from an open vial. In an alternative embodiment, the solvent is allowed to evaporate from a vial covered with a perforated material.

According to another aspect of the present invention, there is provided a process for preparing crystalline Form C of (R)-5-(2-Aminoethyl)-1-(6,8-difluorochroman-3-yl)-1,3-dihydroimidazole-2-thione hydrochloride comprising subjecting Form A or B of (R)-5-(2-Aminoethyl)-1-(6,8-difluorochroman-3-yl)-1,3-dihydroimidazole-2-thione hydrochloride in a solution of ethanol or ethanol/solvent mixtures to evaporation under nitrogen.

According to another aspect of the present invention, there is provided a process for preparing crystalline Faun C of (R)-5-(2-Aminoethyl)-1-(6,8-difluorochroman-3-yl)-1,3-dihydroimidazole-2-thione hydrochloride comprising: (a) stirring a mixture of (R)-5-(2-Aminoethyl)-1-(6,8-difluorochroman-3-yl)-1,3-dihydroimidazole-2-thione hydrochloride in a first organic solvent and an aqueous solution of a base, wherein the first organic solvent is immiscible with water; (b) extracting the organic phase and evaporating the product to dryness; (c) dissolving the product of (b) in dry ethanol; (d) acidifying the product of step (c) with HCl in ethanol; (e) collecting the precipitate; (f) washing the precipitate with ethanol; and (g) drying the product of step (f) to yield Form C.

The first organic solvent may be ethyl acetate. Preferably, the precipitate is collected hot.

In an embodiment, the (R)-5-(2-Aminoethyl)-1-(6,8-difluorochroman-3-yl)-1,3-dihydroimidazole-2-thione hydrochloride is prepared prior to step (a) and converted to Form C in situ by steps (a) to (g). Thus, Form C of compound 1 crystallises and may be isolated, followed by optional recrystallisation to form one of the polymorphic forms.

In an alternative embodiment, the (R)-5-(2-Aminoethyl)-1-(6,8-difluorochroman-3-yl)-1,3-dihydroimidazole-2-thione hydrochloride is prepared prior to step (a), isolated and then converted to Form C by steps (a) to (g).

According to another aspect of the present invention, there is provided a process for preparing crystalline Form C of (R)-5-(2-Aminoethyl)-1-(6,8-difluorochroman-3-yl)-1,3-dihydroimidazole-2-thione hydrochloride comprising slurrying Form A of (R)-5-(2-Aminoethyl)-1-(6,8-difluorochroman-3-yl)-1,3-dihydroimidazole-2-thione hydrochloride in acetonitrile and isolating Form C by vacuum filtration.

In an embodiment, the slurrying is carried out for a period of time ranging from 4 days to 7 days.

According to another aspect of the present invention, there is provided a process for preparing crystalline Form C of (R)-5-(2-Aminoethyl)-1-(6,8-difluorochroman-3-yl)-1,3-dihydroimidazole-2-thione hydrochloride comprising preparing a saturated solution of Form A of (R)-5-(2-Aminoethyl)-1-(6,8-difluorochroman-3-yl)-1,3-dihydroimidazole-2-thione hydrochloride in methanol at an elevated temperature, filtering the warm solution, cooling the solution, and isolating the Form C.

In an embodiment, the cooling brings the temperature of the solution to room temperature.

In another embodiment, the solids are isolated by decantation followed by air drying.

According to another aspect of the present invention, there is provided a process for preparing crystalline Form X of (R)-5-(2-Aminoethyl)-1-(6,8-difluorochroman-3-yl)-1,3-dihydroimidazole-2-thione hydrochloride comprising dissolving Form A of (R)-5-(2-Aminoethyl)-1-(6,8-difluorochroman-3-yl)-1,3-dihydroimidazole-2-thione hydrochloride in methanol, filtering the solution and evaporating the methanol under a stream of nitrogen.

In an embodiment, the evaporation is carried out at about 9% relative humidity.

In another embodiment, the evaporation is carried out at room temperature.

According to another aspect of the present invention, there is provided a pharmaceutical formulation comprising Form A according to the present invention, Form B according to the present invention, Form C according to the present invention or Form X according to the present invention of (R)-5-(2-Aminoethyl)-1-(6,8-difluorochroman-3-yl)-1,3-dihydroimidazole-2-thione hydrochloride and one or more pharmaceutically acceptable carriers or excipients.

According to another aspect of the present invention, there is provided Form A according to the present invention, Form B according to the present invention, Form C according to the present invention or Foam X according to the present invention of (R)-5-(2-Aminoethyl)-1-(6,8-difluorochroman-3-yl)-1,3-dihydroimidazole-2-thione hydrochloride for use in medicine.

According to another aspect of the present invention, there is provided the use of Form A according to the present invention, Form B according to the present invention, Form C according to the present invention or Form X according to the present invention of (R)-5-(2-Aminoethyl)-1-(6,8-difluorochroman-3-yl)-1,3-dihydroimidazole-2-thione hydrochloride in the manufacture of a medicament for treatment of cardiovascular disorders such as congestive heart failure, treatment of angina, treatment of arrhythmias, treatment of circulatory disorders such as Raynaud's Phenomenon (sometimes known as ‘Raynaud's Disease’), treatment of migraine, and treatment of anxiety disorders.

According to another aspect of the present invention, there is provided the use of Form A according to the present invention, Form B according to the present invention, Form C according to the present invention or Form X according to the present invention of (R)-5-(2-Aminoethyl)-1-(6,8-difluorochroman-3-yl)-1,3-dihydroimidazole-2-thione hydrochloride in the manufacture of a medicament for peripherally-selective inhibition of D#H.

In this specification, the term ‘compound 1’ refers to (R)-5-(2-Aminoethyl)-1-(6,8-difluorochroman-3-yl)-1,3-dihydroimidazole-2-thione hydrochloride.

The polymorphs of the present invention are easily prepared and produced in a higher purity than compound 1 synthesised according to the WO2004/033447 process. The purity of the polymorphs of the present invention is particularly advantageous, as it has not previously been possible to achieve the levels of purity which are obtained by the processes of the present invention. Some of the polymorphs are stable to degradation under high humidity for long periods of time.

Reference is made to the accompanying Figures in which:

FIG. 1—XRPD pattern of Form A

FIG. 2—XRPD pattern of Form B

FIG. 3—XRPD pattern of Form C

FIG. 4—XRPD pattern of Form X

FIG. 5—XRPD pattern of amorphous form

FIG. 6—FT-IR spectrum of Form A

FIG. 7—FT-IR spectrum of Form C

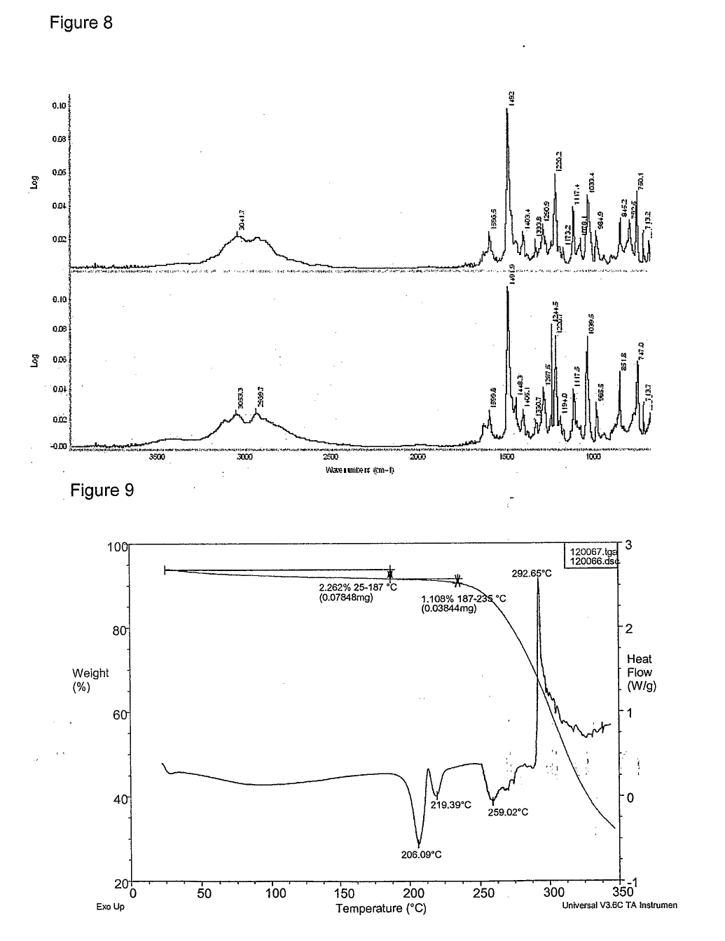

FIG. 8—Stacked FT-IR spectrum of Forms C (top) and A (bottom)

FIG. 9—DSC thermogram for Form A

FIG. 10—DSC thermogram for Form B

FIG. 11—DSC thermogram for Form C

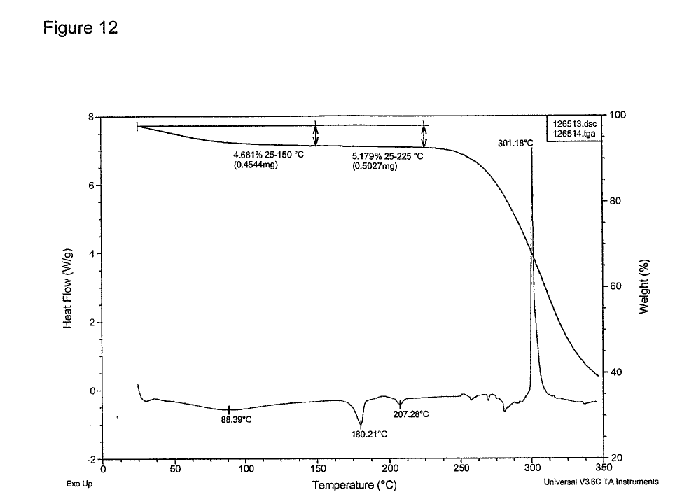

FIG. 12—DSC thermogram for Form X

The analysis of the products of the present invention has shown Faun A to be a variable hydrate with the number of moles of water being dependent on the relative humidity and varying from about 0.09 to about 0.65 moles, Form B to be a variable hydrate with the number of moles of water being dependent on the relative humidity and varying from about 1.1 to about 1.4 moles, Form C to be an anhydrous, unsolvated and non-hygroscopic crystalline solid, and Form X to be crystalline with a disordered crystalline pattern. Form X has also been characterised as a variable hydrate with the number of moles of water varying from 0.26 to 1.85 moles. In an embodiment, Form B is a monohydrate. The chemical structure of all forms was confirmed by ¹H NMR spectroscopy.

Forms B and C of the present invention are advantageous in terms of their stability in that they remain stable under high relative humidities for long periods of time. Furthermore, Form C has been found to be non-hygroscopic.

A further advantage of the polymorphs of the present invention is that they are produced with a high purity, particularly compared to the compound 1 produced according to WO2004/033997. The compound 1 produced according to the WO2004/033997 process has a typical purity of 97%. Typically, the crystalline forms A, B, C and X of the present invention have a purity greater than 97.0% More particularly, the polymorphs have a purity greater than or equal to 97.5%. Advantageously, the polymorphs have a purity greater than or equal to 98.0%. More advantageously, the polymorphs have a purity greater than or equal to 98.5%. Still more advantageously, the polymorphs have a purity greater than or equal to 99.0%. In a preferred embodiment, the polymorphs have a purity greater than or equal to 99.5%.

Form A has been found to be produced by recrystallising compound 1 in aqueous HCl. In an embodiment, the recrystallisation comprises (a) dissolving compound 1 in aqueous HCl, (b) filtering the solution, (c) cooling the solution with stirring, and (d) isolating, washing and drying the precipitated Form A.

Form A may also be produced by forming compound 1 in situ and crystallising

Form A using aqueous HCl. In other words, compound 1 is not isolated to form a solid before being converted to Form A. In an embodiment, the crystallisation comprises (a) adding aqueous HCl to a solution of compound 1, (b) cooling the solution with stirring and (c) isolating, washing and drying the precipitated Form A.

It has also been found that Form A converts to Form B under high laboratory humidity, typically 43% to 90% relative humidity, and particularly, at 55% to 65% relative humidity. The conversion may take place within a time range from 1 day to 2 weeks, and typically after 1 to 2 days. Form B can be dehydrated upon desorption (drying) to convert back to Form A.

Form B has also been found to be produced from vapour stress in ethyl acetate and from experiments using aqueous mixtures of acetone, acetonitrile and ethanol.

Further, Form B has been found to be produced by recrystallising compound 1 from ethanol and toluene.

Form C has been found to be produced by stirring a mixture of compound 1 in a first organic solvent and an aqueous solution of a base, wherein the first organic solvent is immiscible with water; (b) extracting the organic phase and evaporating the product to dryness; (c) dissolving the product of (b) in dry ethanol; (d) acidifying the product of step (c) with HCl in ethanol; (e) collecting the precipitate; (f) washing the precipitate with ethanol; and (g) drying the product of step (f) to yield Form C.

Compound 1 may be isolated before formation of Form C, or compound 1 may be reacted in situ to produce Form C. In other words, compound 1 may be synthesised and converted to Form C without compound 1 being isolated as a solid.

Form C has been found to form during evaporation experiments under nitrogen that used ethanol or ethanol mixtures with other solvents.

Form C was frequently obtained when ethanol, ethyl acetate, and acetonitrile were used for crystallisation.

Form X has been found to be produced by dissolving Form A of compound 1 in methanol, filtering the solution and evaporating the methanol under a stream of nitrogen.

Interconversion studies on Forms A and C in ethanol and acetone:water 99:1 indicated that Form C may be more thermodynamically stable than Form A.

The amorphous form may be prepared by lyophilisation of an aqueous solution of Form A of compound 1.

The amorphous form produced according to the processes of the present invention exhibits higher solubilities in most organic solvents and water compared to Forms A, B, and C of compound 1.

The amorphous form prepared by lyophilisation of Form A will exhibit the same purity as that of the Form A from which it is lyophilised. Thus, the amorphous form prepared in this way will exhibit higher purity than the amorphous form prepared by the WO2004/033447 process.

The invention will now be described with reference to the following non-limiting examples.

### EXAMPLE 1

### Preparation of (R)-5-(2-Aminoethyl)-1-(6,8-difluorochroman-3-yl)-1,3-dihydroimidazole-2-thione hydrochloride Form A

A sample of compound 1 (20 g) prepared according to the method disclosed in WO2004/033447 was stirred in 2N HCl (500 mL) at 75° C. until a clear solution was obtained. The solution was filtered, cooled in the ice bath, and left in the ice bath for 1 h with stirring. Precipitate was collected, washed with cold 2N HCl (ca. 100 mL), cold IPA (ca. 100 mL), dried in vacuum at 40° C. to constant weight. Yield 17.5 g (88%). HPLC purity 99.0%.

### EXAMPLE 2

### Preparation of (R)-5-(2-Aminoethyl)-1-(6,8-difluorochroman-3-yl)-1,3-dihydroimidazole-2-thione hydrochloride Form A

A sample of compound 1 was prepared. Before isolation of the compound 1 to form a solid, 6N HCl (40 mL) was added to a solution of compound 1, the suspension was cooled in ice for 1 h with stirring, the precipitate was collected, washed with cold 3N HCl (75 mL), cold IPA (50 mL), and dried in a vacuum at 50° C. Yield 11.58 g (73%). HPLC purity 99.8%.

### EXAMPLE 3

### Preparation of (R)-5-(2-Aminoethyl)-1-(6,8-difluorochroman-3-yl)-1,3-dihydroimidazole-2-thione hydrochloride Form B

A sample of Form A of compound 1 (1 g) was kept at 65% relative humidity and 25° C. for 48 h. Yield 1.05 g. HPLC purity 99.8%.

Karl Fisher analysis showed Form B to contain approximately 6.6% or 1.3 moles of water.

Form B was found to be stable at 90% relative humidity and no deliquescence was observed at 90% relative humidity after 10 days.

Further preparations of Form B were carried out at varying relative humidities. The number of moles of water depended on the relative humidity and varied from about 1.1 to about 1.4 moles.

Thus, Form B is a variable hydrate of compound 1.

### EXAMPLE 4

### Preparation of (R)-5-(2-Aminoethyl)-1-(6,8-difluorochroman-3-yl)-1,3-dihydroimidazole-2-thione hydrochloride Form C

Form A as prepared above, or compound 1 prepared according to the method disclosed in WO2004/033997 was stirred in the mixture of ethyl acetate (150 mL) and 10% NaHCO₃ solution in water for 15 min at room temperature. Organic phase was separated, evaporated to dryness under reduced pressure, the residue was taken up into dry ethanol (100 mL). The solution was acidified with 3M HCl in ethanol to pH 2 and stirred at 65-70° C. for 2 h. The precipitate was collected hot, washed with ethanol dried in vacuum at 40° C. to constant weight. Yield 8.24 g (82%). HPLC purity 99.5%.

### EXAMPLE 5

### Preparation of (R)-5-(2-Aminoethyl)-1-(6,8-difluorochroman-3-yl-1,3-dihydroimidazole-2-thione hydrochloride Form C

The free base of compound 1 produced in situ was dissolved with heating in a mixture of absolute EtOH (15 mL) and 3M HCl in absolute EtOH (1.5 mL, pH of the mixture approximately 2). The resulting solution was stirred at 65-70° C. for 2 hours, the crystals were collected, washed with EtOH, and dried in vacuum at 40° C. Yield 1.12 g (71%). HPLC purity 99.5%.

### EXAMPLE 6

### Preparation of (R)-5-(2-Aminoethyl)-1-(6,8-difluorochroman-3-yl)-1,3-dihydroimidazole-2-thione hydrochloride Form C

Form A was stirred in acetonitrile at room temperature for 96 h under nitrogen. Solid was collected, dried in vacuum at 40° C. to constant weight. Yield 1.8 g (90%). HPLC purity 99.8%.

Form C did not deliquesce after one week at approximately 65% relative humidity, nor after 11 days at approximately 90% relative humidity.

### EXAMPLE 7

### Preparation of (R)-5-(2-Aminoethyl)-1-(6,8-difluorochroman-3-yl)-1,3-dihydroimidazole-2-thione hydrochloride Form X

Form A of compound 1 (200 mg) was dissolved in methanol (5 mL), the solution was filtered through a 0.2-μm nylon filter and evaporated at room temperature under a stream of nitrogen (ca 9% relative humidity).

### EXAMPLE 8

### Crystallisation Experiments on Forms A, B, C, X and the Amorphous Form

Crystallisation experiments were carried out on the different Forms of compound 1. The methods used were slurrying, fast evaporation, slow evaporation, crash cooling, c rash precipitation and slow cool, as described below. Interconversion experiments were also undertaken.

Slurrying

Slurries of compound 1 were prepared by adding enough solids to a given solvent at ambient so that undissolved solids were present. The mixture was then loaded on a rotary wheel or an orbit shaker in a sealed vial at either ambient temperature or elevated temperature for a certain period of time, typically 7 days. The solids were isolated by vacuum filtration or by drawing the liquid phase off with a pipette and allowing the solids to air dry at ambient conditions prior to analysis.

Fast Evaporation

Solutions of compound 1 were prepared in various solvents in which samples were vortexed or sonicated between aliquot additions. Once a mixture reached complete dissolution, as judged by visual observation, the solution was filtered through a 0.2-μm nylon filter. The filtered solution was allowed to evaporate at ambient temperature in an open vial. The solids were isolated and analyzed.

Slow Evaporation

Solutions of compound 1 were prepared in various solvents in which samples were vortexed or sonicated between aliquot additions. Once a mixture reached complete dissolution, as judged by visual observation, the solution was filtered through a 0.2-μm nylon filter. The filtered solution was allowed to evaporate at ambient in a vial covered with aluminum foil perforated with pinholes. The solids were isolated and analyzed.

Crash Cooling

A saturated solution of compound 1 was prepared in methanol at an elevated temperature and filtered warm through a 0.2-μm nylon filter into an open vial while still warm. The vial was capped and cooled to room temperature. Solids were isolated by decanting the solvent and allowed to air-dry prior to analysis.

Crash Precipitation (CP)

Saturated solutions of compound 1 were prepared in various solvents and filtered through a 0.2-μm nylon filter into an open vial. Aliquots of various antisolvents were dispensed until precipitation occurred. Solids were collected by vacuum filtration or by drawing solvent off with a pipette and allowing the solids to air dry at ambient conditions prior to analysis.

Slow Cool (SC)

Saturated solutions of compound 1 were prepared in various solvents at an elevated temperature and filtered warm through a 0.2-μm nylon filter into an open vial while still warm. The vial was capped and left on the hot plate, and the hot plate was turned off to allow the sample to slow cool to ambient temperature. The solids were isolated by vacuum filtration and analyzed.

Interconversion Experiments

A slurry of compound 1, Form A, was prepared in a given solvent and loaded on an orbit shaker at either ambient or elevated temperature for at least 1 day. The liquid phase of the slurry was drawn off with a pipette and filtered through a 0.2-μm filter. A slurry containing compound 1 forms A and C was prepared using the filtered liquid phase from the Form A slurry. The mixture was then loaded on an orbit shaker in a sealed vial at either ambient or elevated temperature for 1 or 7 days. The solids were isolated by vacuum filtration or by drawing the liquid phase off with a pipette and allowing the solids to air dry at ambient conditions prior to analysis.

The resulting Forms were characterised by XRPD.

A capillary screen was conducted on Form A and the amorphous form using solvent/antisolvent crystallisations and vapor stress experiments. Various crystallisation techniques were employed. These techniques are described below. X-ray powder diffraction quality capillaries were used. Once solids were observed from the crystallisation attempts, they were examined under a microscope for birefringence and morphology. Any crystalline shape was noted, but sometimes the solid exhibited unknown morphology, in some cases due to the packing in the capillary or to small particle size. When sufficient, solid samples were then analyzed by XRPD.

CentriVap Crystallisations—Form A

A solution of Form A in a given solvent at an approximate concentration of 87 mg/mL was prepared and filtered through a 0.2-μm filter. A capillary was filled with 15 μL of solution, and then 25 μL of an antisolvent was added. The capillary was centrifuged. The solvent was evaporated in a Labconco CentriVap® centrifugal evaporator under reduced pressure using a mechanical vacuum pump. The evaporator temperature was maintained at ambient temperature.

CentriVap Crystallisations (CentriVap)—Amorphous form

A solution of compound 1, amorphous, in a given solvent was prepared and filtered through a 0.2-μm filter. A capillary was filled with 45 μL of solution via syringe. The capillary was centrifuged. The solvent was evaporated in a Labconco CentriVap® centrifugal evaporator under reduced pressure using a mechanical vacuum pump. The evaporator temperature was maintained at ambient temperature.

Crystallisations by Fast Evaporation

A solution of Form A in a given solvent at an approximate concentration of 87 mg/mL was prepared and filtered through a 0.2-μm filter. A capillary was filled with 15 μl, of solution, and then 25 μL of an antisolvent was added. The capillary was centrifuged. Evaporations were performed in open capillaries at ambient temperature.

Evaporation in Capillary (EC)

A solution of compound 1, amorphous, in a given solvent was prepared and filtered through a 0.2-μm filter. A capillary was filled with 45 μL of solution via syringe. The capillary was centrifuged. Evaporations were preformed in open capillaries at ambient and elevated temperatures or under nitrogen flow with low (approximately 19%) relative humidity at ambient temperature.

Solvent/Antisolvent Crystallisations in Capillary

A solution of compound 1, amorphous, in a given solvent was prepared and filtered through a 0.2-μm filter. A capillary was filled with 15 μL of solution and centrifuged down. Then 30 μL of an antisolvent was added. The capillary was centrifuged. If a clear solution resulted the capillary was left at ambient to allow the solvents to evaporate or evaporation was performed in a Labconco Centrivap centrifugal evaporator under reduced pressure using mechanical pump at ambient. Evaporation was carried out also under nitrogen flow with low (approximately 19%) relative humidity.

Vapor Diffusion into Solid or Vapor Stress (VS)—Form A

Capillaries were packed with approximately 1 cm of Form A. The capillaries were placed in tall vials containing about 5 mL of solvents or solvent mixtures. The capillaries were removed after approximately 8 days.

Vapor Diffusion into Solid or Vapor Stress (VS)—Amorphous Form

Capillaries were packed with approximately 1 cm of compound 1, amorphous. The solids were exposed to vapor diffusion by placing the capillaries in tall vials containing about 5 mL of solvents. The capillaries were removed after approximately 10 days.

### Results

### Polymorph Screen of Compound 1, Form A

Approximate solubilities of compound 1, Form A in aqueous mixtures containing high concentrations of organic solvents are given in Table 1.

TABLE 1
Approximate Solubilities of compound 1, Form A

| Solvent | Solubility (mg/mL)^{a} |
|---|---|
| acetone:water 90:10 | >23 |
| acetone:water 99:1 | 1 |
| acetonitrile:water 90:10 | >23 |
| acetonitrile:water 95:5 | <23 |
| 1,4-dioxane:water 99:1 | <24 |
| ethanol:water 90:10 | >21 |
| ethanol:water 99:1 | <25 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 2
Crystallisation Results on compound 1, Form A

| Solvent | Method^{a} | Observations | XRPD Results |
|---|---|---|---|
| ethyl ether | slurry | light yellow solid | B |
| hexane | slurry | light yellow solid | B |
| acetonitrile | slurry, 7 d | white solid | C |
| | slurry, 4 d | white solid | C |
| 1,4-dioxane | slurry | viscous yellow oil | — |
| ethanol | FE | white solid | B |
| ethyl acetate | slurry | white solid | B |
| hexafluoroisopropanol | FE | white solid | B |
| methanol | CC | white solid | C |
| | FE | white solid, | B |
| | | dendridic | |
| | | formations | |
| methylene chloride | slurry | white solid | B |
| methyl ethyl ketone | slurry | light yellow solid | A |
| toluene | slurry | white solid | B |
| propionitrile | slurry | white solid | B |
| trifluorotoluene | slurry | white solid | B |
| ethanol:acetonitrile | SE | light yellow solid | B |
| 1:1 | | | |
| ethanol:1,4-dioxane | SE | yellow oil | — |
| 1:1 | | | |
| ethanol:ethyl | SE | light yellow solid | B |
| acetate 1:1 | | | |
| ethanol:methyl | SE | light brown oil | — |
| ethyl ketone 1:1 | | | |
| ethanol:toluene 1:1 | SE | light yellow solid | B |
| ethanol:cyclohexane | SE | white solid | B |
| 1:1 | | | |
| ethanol:methyl iso- | SE | light beige solid | B |
| butyl ketone 1:1 | | | |
| ethanol:n-butyl | SE | light yellow solid | B |
| acetate 1:1 | | | |
| ethanol | SE | white solid | B |
| methanol:methylene | SE | white solid | B |
| chloride | | | |
| methanol:acetone | SE | white solid | B |
| methanol:acetonitrile | SE | white solid | B |
| methanol:1,4- | SE | light yellow solid | low crystalline B |
| dioxane | | | |
| methanol:ethyl | SE | white solid | B |
| acetate | | | |
| methanol:methyl | SE | pink solid^{b} | low crystalline B |
| ethyl ketone | | | |
| methanol:toluene | SE | white solid | B |
| methanol:cyclohexane | SE | white solid | B |
| methanol:iso-propyl | SE | white solid | B |
| ether | | | |
| methanol | SE | white solid | B |

^{a}FE = fast evaporation, SE = slow evaporation, CC = crash cooling; times are approximate
^{b}possible impurity/degradation

As the initial polymorph screen results were affected by the laboratory humidity, additional crystallisation experiments were carried out under nitrogen at approximately 9% relative humidity. Methanol, ethanol, and their mixtures with other solvents were used.

TABLE 3
Crystallisation by evaporation under nitrogen

| | | XRPD |
|---|---|---|
| Solvent/Solvent Mixture | Observations | Result |
| methanol | white solid | X |
| methanol:acetone 1:1 | white solid | X |
| methanol:ethyl acetate 1:1 | white solid | X |
| methanol:isopropyl ether | white solid | X |
| 1:1 | | |
| methanol:methyl iso-butyl | light yellow solid | Amorphous |
| ketone 1:1 | | |
| methanol:n-butyl acetate | white solid, wet | X |
| 1:1 | | |
| Ethanol | white solid | C |
| Ethanol:acetonitrile 1:1 | white solid | C |
| ethanol:ethyl acetate 1:1 | white solid | C |
| ethanol:propionitrile 1:1 | light yellow solid | C |
| ethanol:iso-propyl acetate | white solid | C |
| 1:1 | | |

TABLE 4
Capillary screen of Form A by solvent/antisolvent crystallisation

| | | | | XRPD |
|---|---|---|---|---|
| Solvent | Antisolvent | Method^{a} | Morphology | Result^{f} |
| MeOH | acetone | FE | White, | C + B |
| | | | morphology | l.c. |
| | | | unknown, | |
| | | | not birefringent | |
| | | CentryVap | White, | X |
| | | | broken | |
| | | | glass, not birefringent | |
| | acetonitrile | FE | White, | B |
| | | | morphology | |
| | | | unknown, | |
| | | | not birefringent | |
| | | CentryVap | White, | X |
| | | | morphology | |
| | | | unknown, | |
| | | | not birefringent | |
| | methyl ethyl | FE | Pink | — |
| | ketone | | needles, birefringent^{b} | |
| | | CentryVap | White, small | B |
| | | | part | |
| | | | crystalline, | |
| | | | birefrigent, | |
| | | | mostly glass, | |
| | | | not birefringent | |
| | ethyl | Precipitation | White, | amorphous + |
| | acetate | | dendridic | C peaks |
| | | | formations, | |
| | | | birefringent^{c} | |
| | | Precipitation | White, | C |
| | | | morphology | |
| | | | unknown, | |
| | | | not birefringent | |
| | CH₂Cl₂ | Precipitation | White, tiny | C |
| | | | plates and | |
| | | | needles, birefringent | |
| | | Precipitation | White, tiny | C |
| | | | plates, birefringent | |
| | methyl t- | Precipitation | White, | B |
| | butyl ether | | dendridic | |
| | | | and tiny | |
| | | | plates, birefringent | |
| | | Precipitation | White, | C + B |
| | | | dendridic | l.c. |
| | | | and plates, | |
| | | | birefringent | |
| | iso-propyl | Precipitation | White, | C |
| | acetate | | morphology | |
| | | | unknown, | |
| | | | not birefringent | |
| | | Precipitation | White, | B |
| | | | irregular and | |
| | | | tiny, birefringent | |
| | tetrahydrofuran | FE | Off-white, | low |
| | | | morphology | crystalline |
| | | | unknown, | B, shifted |
| | | | not birefringent | |
| | | CentryVap | White, | amorphous |
| | | | morphology | or no solid |
| | | | unknown, | |
| | | | not birefringent | |
| | toluene | Precipitation | White, | amorphous + |
| | | | morphology | C peaks |
| | | | unknown, | |
| | | | birefringent^{d} | |
| | | Precipitation | White, | C (l.c.) |
| | | | morphology | |
| | | | unknown, in | |
| | | | solution, birefringent | |
| | 2,2,2-triflouro-ethanol | FE | Needles on | — |
| | | | sides of | |
| | | | capillary^{e}, | |
| | | | birefringent; | |
| | | | clear viscous | |
| | | | liquid on | |
| | | | bottom | |
| CentryVap | | White, | C + B | |
| | | morphology | l.c. | |
| | | unknown, | | |
| | | not birefringent | | |

^{a}FE = fast evaporation, CentryVap = evaporation under reduced pressure using centrifugal evaporator
^{b}pink solid (possible impurity/degradation)
^{c}the solid only on the sides of capillary
^{d}the solid moved from the originally marked spot
^{e}insufficient for XRPD analysis
^{f}l.c. = low crystalline, i.e. crystalline product having a disordered crystalline pattern

TABLE 5
Vapour stress on Form A

| | | XRPD |
|---|---|---|
| Solvent | Morphology | Result |
| EtOAc | White, morphology | B |
| | unknown, not birefringent | |
| i-PrOH | White, morphology | A |
| | unknown, not birefringent | |
| acetone:water 1:1 | White, morphology | B |
| | unknown, not birefringent | |
| acetonitrile:water 4:1 | White, morphology | B |
| | unknown, not birefringent | |
| ethanol:water 1:1 | White, morphology | B |
| | unknown, not birefringent | |

Thus, Form A remained unchanged under iso-propanol vapor. Form B resulted from a vapor stress in ethyl acetate (possibly due to the affect of laboratory humidity). Form B also resulted from experiments using aqueous mixtures of acetone, acetonitrile and ethanol.

A further, abbreviated polymorph screen was carried out on Form A by fast evaporation and slimy experiments at ambient temperature and 40° C. The results for the screen are summarized in Table 6 and Table 7. Forms A, B, C, and amorphous material were all produced from these experiments. Whether Form A or Form B was produced is thought to depend on the drying conditions and laboratory humidity.

Form B resulted from t-butyl methyl ether, acetone:water 99:1, and iso-propanol: water 90:10 slurries, likely due to laboratory humidity or drying conditions.

Form C was often produced when ethanol, ethyl acetate, and acetonitrile were used for crystallisation.

Form X was often produced from methanol solutions.

TABLE 6
Crystallisation Experiments on compound 1, Form A

| Solvent | Conditions^{a} | Habit/Description | XRPD Result |
|---|---|---|---|
| t-butyl methyl ether | slurry, | white solid | A |
| | 3 days | | |
| dichloromethane | slurry, | white, morphology | A |
| | 7 days | unknown, not birefringent | |
| ethyl acetate:hexane | slurry, | white, morphology | A |
| 1:1 | 7 days | unknown, partially | |
| | | birefringent | |
| acetone:water | FE | white, morphology | B |
| 90:10 | | unknown, partially | |
| | | birefringent | |
| acetone:water | slurry, | white, morphology | A |
| 99:1 | 7 days | unknown, not birefringent | |
| | FE (filtrate from | clear glassy slurry) | — |
| | | irregular particles, | |
| | | partially birefringent | |
| acetonitrile:water | FE | white, morphology | A + B (minor) |
| 90:10 | | unknown, not birefringent | |
| acetonitrile:water | slurry, | white plates, birefringent | C |
| 95:5 | 7 days | | |
| | FE (liquid phase from | white spherulites of | — |
| | slurry) | fibers, birefringent | |
| 1,4-dioxane:water | slurry, | light pink, | A |
| 99:1 | 7 days | morphology | |
| | | unknown, not birefringent | |
| | FE (liquid phase | yellow glassy film, | — |
| | from slurry) | not birefringent; | |
| | | yellow, morphology | |
| | | unknown, birefringent | |
| ethanol:water | FE | white fibers, birefringent | B + A (minor) |
| 90:10 | | | |
| ethanol:water | slurry, | white blades, birefringent | C |
| 99:1 | 7 days | white, morphology | C + A (minor) |
| | | unknown, partially | |
| | | birefringent | |
| | FE (liquid phase | white fibers, birefringent | amorphous |
| | from slurry) | | |
| isopropanol:water | slurry, | white, morphology | B |
| 90:10 | 7 days | unknown, not birefringent | |
| 0.1 N HCl | FE, ambient | white, needles, birefringent | B |
| | FE under N₂ | white, dendridic | B |
| | ~12% RH | formations, birefringent | |

TABLE 7
Crystallisation Experiments on compound 1 Form A, at 40° C.

| | | | XRPD |
|---|---|---|---|
| Solvent | Conditions | Habit/Description | Result^{a} |
| t-butyl methyl | slurry, 40° C., | off-white solid | B |
| ether | 3 days | | |
| ethanol | slurry, 40° C., | white blades, birefringent | C |
| | 7 days | | |
| ethyl acetate | slurry, 40° C., | white, morphology | C |
| | 7 days | unknown, birefringent | |
| | | white, morphology | B + C |
| | | unknown, partially | |
| | | birefringent | |
| ethyl | slurry, 40° C., | white, morphology | B + A |
| acetate:hexane | 7 days | unknown, not birefringent | (minor) |
| 1:1 | | | |
| methyl ethyl | slurry, 40° C., | white, morphology | A, l.c. |
| ketone | 7 days | unknown, not birefringent | |
| toluene | slurry, 40° C., | white, morphology | A |
| | 7 days | unknown, not birefringent | |
| acetone:water | slurry, 40° C., | white, morphology | B |
| 99:1 | 7 days | unknown, not birefringent | |
| acetonitrile:water | slurry, 40° C., | white plates, birefringent | C |
| 95:5 | 7 days | | |
| ethanol:water | slurry, 40° C., | white needles and | C, l.c. |
| 99:1 | 7 days | blades, birefringent | |
| isopropanol:water | slurry, 40° C., | off-white dendridic | B |
| 9:1 | 7 days | needles, birefringent | |

^{a}l.c. = low crystallinity

Polymorph Screen of Compound 1, Form B

Form B showed higher solubilities in aqueous solvent mixtures compared to organic solvents (Table 8).

TABLE 8
Approximate Solubilities of compound 1, Form B^{a}

| Solvent | Solubility (mg/mL)^{b} |
|---|---|
| acetone | 2 |
| ethanol | 4 |
| methanol | 15 |
| acetone:water 99:1 | <29 |
| acetonitrile:water 95:5 | <27 |
| ethanol:water 99:1 | <28 |

^{a}prepared from Form A at 90% RH
^{b}Solubilities are calculated based on the total solvent used to give a solution; actual solubilities may be greater because of the volume of the solvent portions utilized or a slow rate of dissolution. Solubilities are reported to the nearest mg/mL.

An abbreviated polymorph screen was carried out on Form B by slow evaporation and slurry experiments at ambient conditions, 40° C., and lower relative humidities under nitrogen gas (approximately 12-20% RH). The results of the screen are summarized in Tables 9, 10 and 11. Forms A, B, C, and X were all produced.

Forms A, B, and a mixture of Forms B and A were isolated from ambient slurries in acetone, and aqueous acetone, acetonitrile and ethanol (Table 9). It is thought that Forms A and B resulted from different drying conditions.

TABLE 9
Crystallisation Experiments on compound 1, Form B^{a}

| Solvent | Conditions | Habit/Description | XRPD Result |
|---|---|---|---|
| acetone | slurry, | clear solution | — |
| | 3 days | | |
| acetone:water 99:1 | slurry, | white, morphology | B |
| | 7 days | unknown, not | |
| | | birefringent | |
| acetonitrile:water | slurry, | white, morphology | B + A |
| 95:5 | 7 days | unknown, partially | |
| | | birefringent | |
| ethanol:water 99:1 | slurry, | white, morphology | A |
| | 7 days | unknown, not | |
| | | birefringent; white | |
| | | fibers, birefringent | |

^{a}prepared from Form A at 90% RH

TABLE 10
Crystallisation Experiments on compound 1, Form Ba,
by Evaporation under Nitrogen (~20% RH)

| Solvent | Conditions^{a} | Habit/Description | XRPD Result |
|---|---|---|---|
| acetone | SE | — | — |
| ethanol | SE | white flakes, not birefringent | C |
| methanol | SE | white needles, birefringent | X |

^{a}prepared from Form A at 90% RH
b. SE = slow evaporation
c. Samples were exposed to ambient conditions due to a gap in nitrogen gas flow.

TABLE 11
Crystallisation Experiments on compound 1, Form B^{a}, at 40° C.

| Solvent | Conditions | Habit/Description | XRPD Result |
|---|---|---|---|
| ethanol | slurry, 40° C., | white blades and | C |
| | 7 days | needles, birefringent | |
| ethyl acetate | slurry, 40° C., | white, morphology | A |
| | 7 days | unknown, not birefringent | |
| methyl ethyl ketone | slurry, 40° C., | white, morphology | A |
| | 7 days | unknown, not birefringent | |
| toluene | slurry, 40° C., | white, morphology | B + A (very minor) |
| | 7 days | unknown, not birefringent | |
| acetone:water 99:1 | slurry, 40° C., | white and yellow, | A |
| | 3 days | morphology | |
| | | unknown, not birefringent | |
| acetonitrile:water | slurry, 40° C., | white plates and | C + B (very minor) |
| 95:5 | 7 days | blades, birefringent | |
| ethanol:water 99:1 | slurry, 40° C., | white blades, birefringent; | C |
| | 7 days | white | |
| | | fibers, partially birefringent | |

^{a}prepared from Form A at 90% RH

Polymorph Screen of Compound 1, Form C

Approximate solubilities of compound 1, Form C are given in Table 12. The material was poorly soluble in most organic solvents, except in methanol. It was slightly soluble in ethanol, hexafluoroisopropanol, 2,2,2-trifluoroethanol, and some aqueous mixtures. Overall, the solubilities of Form C were lower compared to the solubilities of Form A.

TABLE 12
Approximate Solubilities of compound 1 Form C

| Solvent | Solubility (mg/mL)^{a} |
|---|---|
| acetone | <1 |
| acetonitrile | <1 |
| dichloromethane | <1 |
| diethyl ether | <1 |
| 1,4-dioxane | <1 |
| ethanol | 2 |
| ethyl acetate | <1 |
| hexafluoro isopropanol | 1 |
| hexane | <1 |
| iso-propanol | <1 |
| methanol | 21 |
| methyl ethyl ketone | <1 |
| n-propanol | <1 |
| tetrahydrofuran (THF) | <1 |
| toluene | <1 |
| 2,2,2-trifluoroethanol | 1.7 |
| water | 23 |
| acetone:water 9:1 | >23 |
| acetone:water 99:1 | <24 |
| acetonitrile:water 4:1 | >40 |
| acetonitrile:water 9:1 | <25 |
| 1,4-dioxane: water 9:1 | >22 |
| 1,4-dioxane:water 99:1 | <22 |
| ethanol:water 9:1 | <22 |
| THF:water 9:1 | >23 |
| THF:water 99:1 | <24 |

^{a}Solubilities are calculated based on the total solvent used to give a solution; actual solubilities may be greater because of the volume of the solvent portions utilized or a slow rate of dissolution. Solubilities are reported to the nearest mg/mL.

A polymorph screen was carried out on Form C by fast and slow evaporation, crash precipitation, crash cooling, slow cooling, rotary evaporation, and slurry experiments at ambient and lower relative humidity conditions. The results of the screen are summarized in Table 13 and Table 14. Forms A, B, C, X, and amorphous material were all produced.

Form B or low crystalline Form B resulted from most of the fast evaporation experiments. Form B was also produced from crash cooling and slow cooling experiments in water. Low crystalline Form B resulted from slow evaporation in acetone: methanol 4:1.

Form C remained unchanged in all the slurry experiments. Forms A, B, or the amorphous form were isolated from solution-based crystallisations. Form C also resulted from slow evaporation experiments in 4:1 acetonitrile:methanol and ethyl acetate: methanol, and from rotary evaporation in ethanol. This is similar to results discussed above from the polymorph screens of Forms A and B, where experiments utilizing acetonitrile, ethyl acetate, and ethanol most often produced Form C.

Form X was mostly produced from experiments utilizing methanol.

Based on XRPD, Form C did not change after 2 months at 95% relative humidity.

TABLE 13
Crystallisation Experiments on compound 1 Form C

| Solvent | Conditions^{a} | Habit/Description | XRPD Result^{b} |
|---|---|---|---|
| acetone | slurry, | white solid | C |
| | 8 days | | |
| acetone:methanol | SE | white, morphology | B, small amount of |
| 4:1 | | unknown, not birefringent | sample |
| acetonitrile | slurry, | white solid | C |
| | 8 days | | |
| acetonitrile:methanol | SE | white dendridic | C |
| 4:1 | | formations, birefringent; | |
| | | light | |
| | | brown, morphology | |
| | | unknown, not birefringent | |
| dichloromethane | slurry, | white solid | C |
| | 7 days | | |
| diethyl ether | slurry, | white, morphology | C |
| | 7 days | unknown, not birefringent | |
| | FE (liquid phase | clear glassy film, | — |
| | from slurry) | not birefringent | |
| | CP w/methanol | white spherulites of | amorphous with |
| | | fibers and | peaks from X |
| | | morphology | |
| | | unknown, birefringent | |
| 1,4-dioxane | slurry, | white solid | C |
| | 8 days | | |
| ethanol | FE | white spherulites of | B |
| | | needles, birefringent | |
| | SC | clear solution | — |
| | RE | off-white, | C, l.c. |
| | | morphology | |
| | | unknown, not birefringent | |
| ethyl acetate | slurry, | white solid | C |
| | 8 days | | |
| ethyl acetate:methanol | SE | white fibers, not birefringent; | C |
| 4:1 | | yellow, | |
| | | morphology | |
| | | unknown, birefringent | |
| hexafluoro iso- | FE | white fibers, | B |
| propanol | | partially birefringent | |
| hexane | slurry, | white, morphology | C + peaks |
| | 7 days | unknown, not birefringent | |
| | FE (liquid phase | translucent oily film, | — |
| | from slurry) | not birefringent | |
| hexane:methanol | stand (capped) at | clear solution, two | — |
| 2:1 | ambient conditions | layers present; | |
| | | sample was | |
| | | discarded | |
| iso-propanol | slurry, | white solid | C |
| | 8 days | | |
| methanol | FE | white, dendridic | B, small amount of |
| | | formations, birefringent | material |
| | CC | white, morphology | X |
| | | unknown, birefringent | |
| | SC | clear solution | — |
| | RE | off-white, | X |
| | | morphology | |
| | | unknown, partially | |
| | | birefringent | |
| methyl ethyl ketone | slurry, | white solid | C |
| | 8 days | | |
| methyl ethyl ketone:methanol | SE | dark red viscous | — |
| 4:1 | | liquid, not birefringent; | |
| | | yellow, | |
| | | morphology | |
| | | unknown, birefringent | |
| n-propanol | slurry, | white, morphology | C |
| | 7 days | unknown, not birefringent | |
| | FE (liquid phase | light brown | X |
| | from slurry) | spherulites of fibers, | |
| | | birefringent | |
| | FE (sat. solution) | white solid | X |
| tetrahydrofuran | slurry, | white solid | C |
| (THF) | 8 days | | |
| toluene | slurry, | white solid | C |
| | 8 days | | |
| toluene:methanol | SE | white fibers and | X, l.c. |
| 4:1 | | needles, partially birefringent | |
| 2,2,2-trifluoroethanol | FE | white, morphology | B, small amount of |
| | | unknown | material |
| water | FE | white dendridic | A |
| | | formations, birefringent | |
| | CC | white solid | B |
| | SC | white solid | B |
| acetone:water 9:1 | FE | white, morphology | B |
| | | unknown, partially | |
| | | birefringent | |
| acetone:water 99:1 | slurry, | white, morphology | C |
| | 7 days | unknown, not birefringent | |
| | FE (liquid phase | clear glassy film, | — |
| | from slurry) | not birefringent; | |
| | | clear morphology | |
| | | unknown, birefringent | |
| acetonitrile:water | FE | white dendridic | B |
| 4:1 | | formations, birefringent; | |
| | | white, | |
| | | morphology | |
| | | unknown, not birefringent | |
| | stand (capped) at | clear solution | — |
| | ambient conditions | | |
| | 1 day | | |
| | FE | white with brown at | B |
| | | edge of solid, | |
| | | morphology | |
| | | unknown, not birefringent | |
| acetonitrile:water | slurry, | white needles, birefringent; | C |
| 9:1 | 7 days | white, | |
| | | morphology | |
| | | unknown, not birefringent | |
| | FE (liquid phase | light yellow fibers, | B, small amount of |
| | from slurry) | birefringent; light | material |
| | | yellow, morphology | |
| | | unknown, not birefringent | |
| 1,4-dioxane:water | FE | yellow fibers, birefringent; | amorphous with |
| 9:1 | | yellow, | peaks from X |
| | | morphology | |
| | | unknown, not birefringent | |
| 1,4-dioxane:water | slurry, | yellow, morphology | C |
| 99:1 | 7 days | unknown, not birefringent | |
| | FE (liquid phase | clear glassy film, | — |
| | from slurry) | not birefringent; | |
| | | clear spherulites of | |
| | | fibers, birefringent | |
| ethanol:water 9:1 | slurry, | white, morphology | C |
| | 7 days | unknown, not birefringent | |
| | FE (liquid phase | white spherulites of | B, small amount of |
| | from slurry) | fibers and needles, | material |
| | | birefringent | |
| THF:water 9:1 | FE | yellow, morphology | amorphous with |
| | | unknown, not birefringent | peaks from A |
| THF:water 99:1 | slurry, | yellow, morphology | C, small amount of |
| | 7 days | unknown, partially | material |
| | | birefringent | |
| | FE (liquid phase | clear, morphology | — |
| | from slurry) | unknown, birefringent | |
| — | 95% RH, | — | C |
| | ~2 months | | |

^{a}CC = crash cool, CP = crash precipitation, FE = fast evaporation, RE = rotary evaporation, SC = slow cool, SE = slow evaporation.
^{b}l.c. = low crystallinity.

TABLE 14
Crystallisation Experiments on compound 1 Form C, by Evaporation
under Nitrogen (~17% RH)

| | | Habit/ | |
|---|---|---|---|
| Solvent | Conditions^{a} | Description | XRPD Result |
| 1,4-dioxane:methanol | SE | orange, | amorphous |
| 4:1 | | morphology | |
| | | unknown, not | |
| | | birefringent | |
| tetrahydrofuran:methanol | SE | brown plates, | amorphous |
| 4:1 | | not birefringent | |

^{a}SE = slow evaporation.
b. Sample was exposed to ambient conditions due to a gap in nitrogen gas flow.

Polymorph Screen of Compound 1, Amorphous Material

The amorphous form was reproducibly prepared by lyophilisation (freeze drying) of an aqueous solution of compound 1, Form A (see Table 15).

Amorphous material exhibited higher solubilities in most organic solvents and water compared to Forms A, B, and C (Table 16). Solubilities in ethanol and 2,2,2-trifluoroethanol at elevated temperatures are given in Table 17.

TABLE 15
Preparation of Amorphous compound 1

| Solvent | Method^{a} | Observations | Result |
|---|---|---|---|
| water | FD, | white, morphology | amorphous |
| | 3 days | unknown, not birefringent | |
| water | FD, | white, morphology | amorphous |
| | 3 days | unknown, not birefringent | |
| water | FD, | white solid | amorphous |
| | 4 days | | |

^{a}FD = freeze dry

TABLE 16
Approximate Solubilities of compound 1, Amorphous

| Solvent | Solubility (mg/mL)^{a} |
|---|---|
| acetone | 49 |
| acetonitrile | <1 |
| ethanol | 10 |
| ethyl acetate | <1 |
| iso-propanol | 16 |
| methanol | 50 |
| methyl ethyl ketone | 64 |
| methyl iso-butyl ketone | <1 |
| tetrahydrofuran (THF) | 100 |
| 2,2,2-trifluoroethanol | 8 |
| water | 36 |

^{a}Solubilities are calculated based on the total solvent used to give a solution; actual solubilities may be greater because of the volume of the solvent portions utilized or a slow rate of dissolution. Solubilities are reported to the nearest mg/mL.

TABLE 17
Approximate Solubilities of compound 1, Amorphous,
at Elevated Temperatures

| Solvent | Temperature | Solubility (mg/mL)^{a} |
|---|---|---|
| ethanol | 53° C. | 11 |
| 2,2,2-trifluoroethanol | 50° C. | 8 |

^{a}Solubilities are calculated based on the total solvent used to give a solution; actual solubilities may be greater because of the volume of the solvent portions utilized or a slow rate of dissolution. Solubilities are reported to the nearest mg/mL.

A polymorph screen was carried out on the amorphous material by fast and slow evaporation, crash precipitation, crash cooling, slow cooling, and slurry experiments at ambient temperature and lower relative humidity (Table 18, Table 19). Forms A, B, C, X, and amorphous material were all produced.

At ambient conditions, Farms A and B were produced mostly from evaporation and crash precipitation experiments. It is thought that they resulted from different laboratory humidity and drying conditions.

The results are similar to results discussed above where experiments utilizing ethanol and acetonitrile often produced Form C.

Form X was produced by fast evaporation in 1-propanol at laboratory humidity (approx. 20-50% RH) when the amorphous material was dissolved at a concentration of approximately 3 mg/mL. However, a mixture of Form A and Form C, as a minor component, resulted from the same solvent when the amorphous material was dissolved at a higher concentration of approx. 14 mg/mL.

At lower relative humidities under nitrogen, Form C resulted from fast and slow evaporations in mixtures, most of which contained ethanol (Table 19). Form X was produced from slow evaporation experiments in methanol and mixtures containing methanol, which further supports the previous statement that Form X was frequently produced from methanol solutions.

TABLE 18
Crystallisation Experiments on compound 1, Amorphous Material
(~20-50% RH)

| Solvent | Conditions^{a} | Habit/Description | XRPD Result^{b} |
|---|---|---|---|
| acetone | FE | white, morphology | amorphous |
| | | unknown, not birefringent | |
| | spontaneous precipitation | white, morphology | amorphous + |
| | | unknown, not birefringent | unknown peaks |
| | spontaneous precipitation | white, morphology | C |
| | | unknown, not birefringent | |
| acetonitrile | slurry, | white, morphology | C |
| | 1 day | unknown, not birefringent | |
| | filtrate from slurry | sample discarded | — |
| | CP w/acetone | white, morphology | A + one peak from C |
| | | unknown, partially | |
| | | birefringent | |
| | CP w/methyl ethyl | white flakes, not birefringent | A + peaks from C |
| | ketone | | |
| | CP w/THF | light yellow flakes, | A + B |
| | | birefringent | |
| ethanol | FE | white spherulites, | B |
| | | birefringent | |
| | CC | clear solution | — |
| | SE | white needles, birefringent; | B + C |
| | | white, | |
| | | morphology | |
| | | unknown, not birefringent | |
| | SC | clear solution | — |
| ethyl acetate | slurry, | white plates and | C, l.c. |
| | 4 days | morphology | |
| | | unknown, birefringent | |
| | FE (liquid phase | clear glassy film, | — |
| | from slurry) | not birefringent | |
| | CP w/acetone | white, morphology | A |
| | | unknown, not birefringent | |
| | CP w/methyl ethyl | white, morphology | amorphous |
| | ketone | unknown, not birefringent | |
| | CP w/THF | yellow, morphology | A |
| | | unknown, not birefringent | |
| iso-propanol | FE | white, morphology | amorphous + |
| | | unknown, partially | unknown peaks |
| | | birefringent | |
| | spontaneous crystallisation | light yellow, | C |
| | at 51° C. | morphology | |
| | | unknown, not birefringent | |
| | SC, FE | whites flakes, not | A |
| | (filtrate from | birefringent | |
| | spontaneous recrystallisation | | |
| | at 51° C.) | | |
| methanol | FE | white fibers, birefringent | A |
| methyl ethyl ketone | spontaneous crystallisation | white solid | A + amorphous |
| methyl iso-butyl | slurry, | translucent flake, | — |
| ketone | 4 days | not birefringent | |
| | FE (liquid phase | dark brown viscous | — |
| | from slurry) | liquid, not birefringent | |
| | CP w/THF | yellow flakes, not | B, l.c. |
| | | birefringent | |
| n-propanol - 14 mg/ml | FE | white solid | A + |
| amorphous concentration | | | C (minor) |
| n-propanol - 3 mg/ml | FE | white solid | X |
| amorphous concentration | | | |
| tetrahydrofuran | FE | yellow, morphology | amorphous |
| (THF) | | unknown, not birefringent | |
| | spontaneous precipitation | white, morphology | C + amorphous |
| | | unknown, birefringent | |
| 2,2,2-trifluoroethanol | FE | white fibers, birefringent; | B + |
| | | white, | A (minor) |
| | | morphology | |
| | | unknown, not birefringent | |
| | CC | white, morphology | — |
| | | unknown, birefringent | |
| | SC | white, morphology | — |
| | | unknown, not birefringent | |
| water | FE | white dendridic, birefringent | B |

^{a}CC = crash cool, CP = crash precipitation, FE = fast evaporation, SC = slow cool, SE = slow evaporation.
^{b}l.c. = low crystallinity.

TABLE 19
Crystallisation Experiments on compound 1, Amorphous, by Evaporation
under Nitrogen (~12-20% RH)^{c}

| Solvent | Conditions^{a} | Habit/Description | XRPD Result |
|---|---|---|---|
| acetone | SE | yellow solid, | amorphous |
| | | morphology | |
| | | unknown, not birefringent | |
| | FE | yellow solid, | amorphous |
| | | morphology | |
| | | unknown, not birefringent | |
| acetonitrile:ethanol | SE | white blades and | C |
| 4:1 | | morphology | |
| | | unknown, birefringent | |
| acetonitrile:methanol | SE | white, morphology | C |
| 8:1 | | unknown, birefringent | |
| 2-butanone (MEK) | SE | dark brown solid^{b} | — |
| ethanol | SE | white flakes, not birefringent | C + |
| | | | A (minor) |
| | FE | white fibers, birefringent; | C |
| | | white | |
| | | flakes, not birefringent | |
| ethanol:acetonitrile | SE | white flakes, not birefringent | C |
| 1:1 | | | |
| ethanol:ethyl | SE | white fibers, birefringent | C + peaks from A |
| acetate 1:1 | | | |
| ethyl acetate:ethanol | SE | white, morphology | C |
| 4:1 | | unknown, not birefringent | |
| ethyl acetate:methanol | SE | white, morphology | C |
| 8:1 | | unknown, birefringent | |
| methanol | SE | white fibers, birefringent | X |
| X | | | |
| methanol:acetone | SE | white, morphology | X |
| 1:1 | | unknown, partially | |
| | | birefringent | |
| methanol:acetonitrile | SE | white blades and | C |
| 1:1 | | spherulites of fibers, | |
| | | birefringent | |
| methanol:ethyl | SE | white spherulites, | X |
| acetate 1:1 | | partially birefringent | |
| methanol:methyl | SE | yellow dendridic | X + peak |
| iso-butyl ketone 1:1 | | needles, birefringent | |
| methyl iso-butyl | SE | orange glassy film, | — |
| ketone:acetone 8:1 | | not birefringent; | |
| | | orange, morphology | |
| | | unknown, birefringent | |
| methyl iso-butyl | SE | brown oil, not birefringent | — |
| ketone:ethanol 4:1 | | | |
| methyl iso-butyl | SE | white spherulites of | — |
| ketone:methanol | | needles, birefringent; | |
| 8:1 | | orange | |
| | | glassy film, not birefringent | |
| methyl iso-butyl | SE | orange glassy film, | — |
| ketone:methyl ethyl | | not birefringent; | |
| ketone 8:1 | | clear fibers, birefringent | |
| tetrahydrofuran | SE | yellow, morphology | amorphous |
| (THF) | | unknown, not birefringent | |

^{a}SE = slow evaporation
^{b}Discoloration possibly due to decomposition
^{c}Some samples were exposed to ambient conditions due to a gap in nitrogen gas flow

A capillary polymorph screen was carried out on the amorphous material using evaporation experiments at ambient temperature, 40° C., and lower relative humidity under nitrogen. Solvent/antisolvent crystallisations and vapor stress experiments were also utilized. The results for the screen are summarized in Tables 20, 21 and 22. Forms A, B, C, X, amorphous, and various mixtures of those forms resulted.

TABLE 20
Capillary Polymorph Screen of compound 1, Amorphous

| Solvent | Method^{a} | Habit/Description | XRPD Result^{b} |
|---|---|---|---|
| acetone | EC, ambient | Off-white, | A + C + X |
| | | morphology | |
| | | unknown, not | |
| | | birefringent | |
| | EC, 40° C. | Off-white, | amorphous |
| | | morphology | |
| | | unknown, not | |
| | | birefringent | |
| | EC under N₂ | white, | C |
| | (19% RH) | morphology | |
| | | unknown, not | |
| | | birefringent | |
| | CentriVap | Off-white, | amorphous |
| | | morphology | |
| | | unknown, not | |
| | | birefringent | |
| 2-butanone | EC, ambient | white, | A |
| (MEK) | | morphology | |
| | | unknown, not | |
| | | birefringent | |
| | EC, 40° C. | white, | A |
| | | morphology | |
| | | unknown, not | |
| | | birefringent | |
| | EC under N₂ | white, | A |
| | (12% RH) | morphology | |
| | | unknown, not | |
| | | birefringent | |
| | CentriVap | white, | A |
| | | morphology | |
| | | unknown, not | |
| | | birefringent | |
| MeOH | EC, ambient | White needles, | C |
| | | birefringent | |
| | EC, 40° C. | white, needles, | IS |
| | | birefringent | |
| | EC under N₂ | White, dendridic | IS |
| | (19% RH) | formations, | |
| | | birefrngent | |
| | CentriVap | white, | X, l.c. |
| | | morphology | |
| | | unknown, | |
| | | partially birefringent | |
| tetrahydrofuran | EC, ambient | white, | C |
| | | morphology | |
| | | unknown, not | |
| | | birefringent | |
| | EC, 40° C. | white, | C |
| | | morphology | |
| | | unknown, not | |
| | | birefringent | |
| | EC under N₂ | white, | C, l.c. |
| | (12% RH) | morphology | |
| | | unknown, not | |
| | | birefringent | |
| | CentriVap | white, | A + C |
| | | morphology | |
| | | unknown, not | |
| | | birefringent | |
| water | EC, ambient | off-white, | B, l.c. |
| | | morphology | |
| | | unknown, birefringent | |
| | EC, 40° C. | off-white, | IS |
| | | needles, birefringent | |
| | EC under N₂ | white, | B, l.c. |
| | (19% RH^{d}) | dendridic | |
| | | formations, birefringent | |
| | CentriVap | white, | B |
| | | morphology | |
| | | unknown, birefringent | |

^{a}EC = evaporation in capillary, RH = relative humidity
^{b}IS = insufficient amount for XRPD analysis, l.c. = low crystallinity, PO = preferred orientation
c. XRPD results for LIMS 94755 and LIMS 95240 are non-GMP
^{d}Sample was exposed to ambient conditions due to a gap in nitrogen gas flow.

TABLE 21
Capillary Polymorph Screen of compound 1, Amorphous

| | | | | XRPD |
|---|---|---|---|---|
| Solvent | Antisolvent | Method^{a} | Habit/Description | Result^{b} |
| acetone | acetonitrile | precipitation | white, | C |
| | | | morphology | |
| | | | unknown, | |
| | | | not birefringent | |
| | n-butyl | precipitation | white, | C + A |
| | acetate | | morphology | |
| | | | unknown, | |
| | | | not birefringent | |
| | ethyl acetate | precipitation | white, | C |
| | | | morphology | |
| | | | unknown, | |
| | | | not birefringent | |
| | hexane | precipitation | white, | C + B |
| | | | morphology | |
| | | | unknown, | |
| | | | not birefringent | |
| | isopropyl | precipitation | white, | C, l.c. |
| | acetate | | morphology | |
| | | | unknown, | |
| | | | not birefringent | |
| | MIBK | EC | clear brown | IS |
| | | | glassy solid, | |
| | | | not birefringent | |
| | MIBK | CentriVap | off-white, | amorphous + |
| | | | morphology | unknown |
| | | | unknown, | peaks |
| | | | not birefringent | |
| | MIBK | EC under N₂ | white, | C |
| | | (19% RH) | morphology | |
| | | | unknown, | |
| | | | not birefringent | |
| toluene | precipitation | white, | IS | |
| | | morphology | | |
| | | unknown, | | |
| | | birefringent | | |
| THF | acetonitrile | precipitation | white, | C |
| | | | morphology | |
| | | | unknown, | |
| | | | not birefringent | |
| | n-butyl | precipitation | white, | A |
| | acetate | | morphology | |
| | | | unknown, | |
| | | | birefringent | |
| | ethyl acetate | precipitation | white, | C |
| | | | morphology | |
| | | | unknown, | |
| | | | birefringent | |
| | hexane | precipitation | white, | A |
| | | | morphology | |
| | | | unknown, | |
| | | | not birefringent | |
| | MIBK | EC | white, | A |
| | | | morphology | |
| | | | unknown, | |
| | | | not birefringent | |
| | MIBK | CentriVap | white, | amorphous |
| | | | morphology | |
| | | | unknown, | |
| | | | not birefringent | |
| | toluene | precipitation | white, | A |
| | | | morphology | |
| | | | unknown, | |
| | | | birefringent | |

^{a}XRPD results are non-GMP.

TABLE 22
Capillary Polymorph Screen of compound 1, Amorphous by Vapor Stress

| Solvent | Habit/Description | XRPD Result^{b} |
|---|---|---|
| acetonitrile | white, morphology | C |
| | unknown, not birefringent | |
| n-butyl acetate | white, morphology | A |
| | unknown, not birefringent | |
| ethanol | Broken capillary | — |
| ethyl acetate | white, morphology | C |
| | unknown, not birefringent | |
| heptane | white, morphology | amorphous |
| | unknown, not birefringent | |
| hexane | white, morphology | amorphous |
| | unknown, not birefringent | |
| toluene | white, morphology | amorphous |
| | unknown, not birefringent | |
| 95% RH | white, morphology | B |
| | unknown, not birefringent | |

a. XRPD results are non-GMP.

Experiments Producing Form C

The majority of the experiments that produced Form C from all the compound 1 forms were slurries in different solvents and solvent mixtures (Table 23). Form C was generated in slurries at room temperature and 40° C. from both Forms A and B using ethanol, ethanol:water 99:1, and acetonitrile:water 95:5.

Form C remained unchanged in slurry and slow evaporation experiments involving various solvents and aqueous mixtures. Evaporation of solutions prepared from the amorphous material under nitrogen, as well as slurry, crash precipitation, and capillary evaporation experiments in various solvents and mixtures also resulted in Form C. Amorphous material also spontaneously crystallised to produce Form C in acetone at room temperature and in iso-propanol at 51° C.

TABLE 23
Summary of Experiments Producing compound 1, Form C

| Starting | | |
|---|---|---|
| Form | Solvent | Conditions^{a} |
| A | ethanol | slurry, 40° C., |
| | | 7 days |
| | ethyl acetate | slurry, 40° C., |
| | | 7 days |
| | acetonitrile:water 95:5 | slurry, |
| | | 7 days |
| | acetonitrile:water 95:5 | slurry, 40° C., |
| | | 7 days |
| | ethanol:water 99:1 | slurry, |
| | | 7 days |
| | ethanol:water 99:1 | slurry, 40° C., |
| | | 7 days |
| B | ethanol | slurry, 40° C., |
| | | 7 days |
| | | SE under N₂ |
| | ethanol:water 99:1 | slurry, 40° C., |
| | | 7 days |
| C | acetone | slurry, |
| | | 8 days |
| | acetonitrile | slurry, |
| | | 8 days |
| | acetonitrile:methanol 4:1 | SE |
| | dichloromethane | slurry, |
| | | 7 days |
| | diethyl ether | slurry, |
| | | 7 days |
| | 1,4-dioxane | slurry, |
| | | 8 days |
| | ethyl acetate | slurry, |
| | | 8 days |
| | ethyl acetate:methanol 4:1 | SE |
| | iso-propanol | slurry, |
| | | 8 days |
| | methyl ethyl ketone | slurry, |
| | | 8 days |
| | n-propanol | slurry, |
| | | 7 days |
| | tetrahydrofuran (THF) | slurry, |
| | | 8 days |
| | toluene | slurry, |
| | | 8 days |
| | acetone:water 99:1 | slurry, |
| | | 7 days |
| | acetonitrile:water 9:1 | slurry, |
| | | 7 days |
| | 1,4-dioxane:water 99:1 | slurry, |
| | | 7 days |
| | ethanol:water 9:1 | slurry, |
| | | 7 days |
| | THF:water 99:1 | slurry, |
| | | 7 days |
| amorphous | acetone | spontaneous precipitation |
| | acetonitrile:ethanol 4:1 | SE under N₂ |
| | acetonitrile | slurry, |
| | | 1 day |
| | acetonitrile:methanol 8:1 | SE under N₂ |
| | ethyl acetate | slurry, |
| | | 4 days |
| | ethyl acetate:ethanol 4:1 | SE under N₂ |
| | iso-propanol | spontaneous recrystallisation |
| | | at 51° C. |
| | ethanol | FE under N₂ |
| | methanol:acetonitrile | SE under N₂ |
| | 1:1 | |
| | THF | EC, ambient |
| | | EC, 40° C. |
| | | CP w/ acetonitrile |
| | | CP w/ ethyl acetate |
| | ethanol:acetonitrile 1:1 | SE under N₂ |
| | ethyl acetate:methanol 8:1 | SE under N₂ |

^{a}CP = crash precipitation, EC = evaporation in capillary, FE = fast evaporation, SE = slow evaporation.
b. XRPD results are non-GMP.

Interconversion Studies of Forms A and C

Interconversion studies of Forms A and C in ethanol and acetone:water 99:1 were conducted (Table 24). Form C resulted from a 1-day slurry at 40° C. in ethanol. After one week, Form C resulted at both ambient temperature and 40° C.

A mixture of Forms C and B, with Form B as a minor component, was produced from the 1-day slurries at room temperature in both solvents. A mixture of Forms C and A, with Form A as a minor component, resulted from a 1-day slurry at 40° C. in acetone:water 99:1. Note that Form A did not change in a 1-week slurry in acetone: water 99:1 (Table 6).

The interconversion studies indicate that Form C may be more thermodynamically stable than Form A.

TABLE 24
Interconversion Studies of Forms A and C

| Starting | Solvent/Solvent | | | |
|---|---|---|---|---|
| Forms | System | Method | Time | XRPD Result |
| A + C | EtOH | Slurry, RT | 1 week | C |
| | EtOH | Slurry, RT | 1 day | C + |
| | | | | B (minor) |
| | Acetone:water | Slurry, RT | 1 week | C |
| | 99:1 | Slurry, RT | 1 day | C + |
| | | | | B (minor) |
| | EtOH | Slurry, 40° C. | 1 week | C |
| | EtOH | Slurry, 40° C. | 1 day | C |
| | Acetone:water | Slurry, 40° C. | 1 week | C |
| | 99:1 | Slurry, 40° C. | 1 day | C + |
| | | | | A (one peak) |

Polymorph Characterisation

The polymorphs were characterised by a number of methods, including ¹H NMR, X-ray powder diffraction (XRPD), FT-IR spectroscopy, Differential Scanning Calorimetry (DSC), Karl-Fischer Analysis, Thermogravimetry (TG).

¹H NMR

Solution ¹H nuclear magnetic resonance (NMR) spectra were acquired at ambient temperature with a Varian UNITYINOVA-400 spectrometer at a 1H Larmor frequency of 399.8 MHz. The samples were dissolved in methanol-d4. The spectra were acquired with a 1H pulse width of 8.3-8.4 μs, a 2.50 second acquisition time, a 5 second delay between scans, a spectral width of 6400 Hz with 32000 data points, and 40 or 80 coadded scans. The free induction decay (FID) was processed using the Varian VNMR 6.1C software with 65536 points and an exponential line broadening factor of 0.2 Hz to improve the signal-to-noise ratio. The spectrum was referenced to TMS at 0.0 ppm or solvent at 3.31 ppm (CD3OD).

The structures of the amorphous form and all the polymorphs were found to conform to that of compound 1.

Form X samples generated by evaporation from n-propanol and methanol also showed a residual amount of solvent—from 0.009 to 0.088 moles per one mole of compound 1.

X-Ray Powder Diffraction (XRPD)

The following Shimadzu parameters were used to generate peak lists for Forms A, B and C and X.

| | | | | |
|---|---|---|---|---|
| Measurement | | | | |
| Condition | Form A | Form B | Form C | Form X |

X-ray tube

| | | | | |
|---|---|---|---|---|
| target | Cu | Cu | Cu | Cu |
| voltage | 40.0 (kV) | 40.0 (kV) | 40.0 (kV) | 40.0 (kV) |
| current | 40.0 (mA) | 40.0 (mA) | 40.0 (mA) | 40.0 (mA) |

Slits

| | | | | |
|---|---|---|---|---|
| divergence slit | 1.00000 (deg) | 1.00000 (deg) | 1.00000 (deg) | 1.00000 (deg) |
| scatter slit | 1.00000 (deg) | 1.00000 (deg) | 1.00000 (deg) | 1.00000 (deg) |
| receiving slit | 0.15000 (mm) | 0.15000 (mm) | 0.15000 (mm) | 0.15000 (mm) |

Scanning

| | | | | |
|---|---|---|---|---|
| drive axis | 2Theta/Theta | 2Theta/Theta | 2Theta/Theta | 2Theta/Theta |
| scan range | 2.500-40.000 | 2.500-40.000 | 2.500-40.000 | 2.500-40.000 |
| scan mode | Continuous | Continuous | Continuous | Continuous |
| | Scan | Scan | Scan | Scan |
| scan speed | 3.0000 | 3.0000 | 3.0000 | 3.0000 |
| | (deg/min) | (deg/min) | (deg/min) | (deg/min) |
| sampling pitch | 0.0200 (deg) | 0.0200 (deg) | 0.0200 (deg) | 0.0200 (deg) |
| preset time | 0.40 (sec) | 0.40 (sec) | 0.40 (sec) | 0.40 (sec) |

Data Process Condition

| | | | | |
|---|---|---|---|---|
| Smoothing | [MANUAL] | [AUTO] | [AUTO] | [MANUAL] |
| smoothing | 35 | 19 | 19 | 27 |
| points | | | | |
| B.G. Subtraction | [AUTO] | [AUTO] | [AUTO] | [AUTO] |
| sampling points | 45 | 25 | 21 | 37 |
| repeat times | 30 | 30 | 30 | 30 |
| Ka1-a2 | [MANUAL] | [MANUAL] | [MANUAL] | [MANUAL] |
| Separate | | | | |
| Ka1 a2 ratio | 50.0 (%) | 50.0 (%) | 50.0 (%) | 50.0 (%) |
| Peak Search | [AUTO] | [AUTO] | [AUTO] | [AUTO] |
| differential | 39 | 21 | 19 | 31 |
| points | | | | |
| FWHM | 0.050 (deg) | 0.050 (deg) | 0.050 (deg) | 0.050 (deg) |
| threshold | | | | |
| intensity | 30 (par mil) | 30 (par mil) | 30 (par mil) | 30 (par mil) |
| threshold | | | | |
| FWHM ratio | 2 | 2 | 2 | 2 |
| (n − 1)/n | | | | |
| System Error | [NO] | [NO] | [NO] | [NO] |
| Correction | | | | |
| Precise Peak | [NO] | [NO] | [NO] | [NO] |
| Correction | | | | |

The XRPD peak lists generated were as follows.

TABLE 25
XRPD Peak List for Form A

| Peak No. | Position (°2#) | d-spacing | I (intensity) | I/Io |
|---|---|---|---|---|
| 1 | 4.9 | 17.6 | 30 | 12 |
| 2 | 8.3 | 10.5 | 23 | 9 |
| 3 | 10.8 | 8.1 | 20 | 8 |
| 4 | 12.9 | 6.8 | 50 | 20 |
| 5 | 15.0 | 5.8 | 78 | 30 |
| 6 | 16.2 | 5.4 | 94 | 37 |
| 7 | 16.7 | 5.2 | 15 | 6 |
| 8 | 19.3 | 4.5 | 22 | 9 |
| 9 | 19.8 | 4.4 | 34 | 13 |
| 10 | 20.6 | 4.2 | 15 | 6 |
| 11 | 21.1 | 4.1 | 15 | 6 |
| 12 | 21.8 | 4.0 | 37 | 14 |
| 13 | 22.9 | 3.8 | 46 | 18 |
| 14 | 24.2 | 3.6 | 101 | 39 |
| 15 | 25.1 | 3.5 | 38 | 15 |
| 16 | 26.8 | 3.3 | 256 | 100 |
| 17 | 28.2 | 3.1 | 44 | 17 |
| 18 | 28.7 | 3.0 | 32 | 13 |
| 19 | 29.3 | 3.0 | 26 | 10 |
| 20 | 29.9 | 2.9 | 17 | 7 |
| 21 | 30.6 | 2.9 | 24 | 9 |
| 22 | 32.2 | 2.7 | 46 | 18 |
| 23 | 33.1 | 2.7 | 40 | 16 |
| 24 | 33.9 | 2.6 | 25 | 10 |
| 25 | 34.6 | 2.5 | 8 | 3 |
| 26 | 36.0 | 2.4 | 11 | 4 |
| 27 | 36.7 | 2.4 | 12 | 5 |
| 28 | 38.3 | 2.3 | 17 | 7 |
| 29 | 39.1 | 2.2 | 24 | 9 |

TABLE 26
XRPD Peak List for Form B

| Peak No. | Position (°2#) | d-spacing | I (intensity) | I/Io |
|---|---|---|---|---|
| 1 | 4.8 | 18.3 | 46 | 14 |
| 2 | 8.0 | 10.9 | 20 | 6 |
| 3 | 8.6 | 10.2 | 26 | 8 |
| 4 | 10.8 | 8.1 | 22 | 7 |
| 5 | 12.7 | 6.9 | 44 | 13 |
| 6 | 13.2 | 6.6 | 11 | 3 |
| 7 | 13.6 | 6.4 | 36 | 11 |
| 8 | 14.4 | 6.1 | 49 | 15 |
| 9 | 15.2 | 5.7 | 42 | 13 |
| 10 | 15.6 | 5.6 | 32 | 10 |
| 11 | 16.0 | 5.5 | 110 | 34 |
| 12 | 19.3 | 4.5 | 23 | 7 |
| 13 | 19.8 | 4.4 | 31 | 10 |
| 14 | 20.3 | 4.3 | 17 | 5 |
| 15 | 20.6 | 4.3 | 16 | 5 |
| 16 | 21.2 | 4.1 | 17 | 5 |
| 17 | 21.7 | 4.0 | 49 | 15 |
| 18 | 22.9 | 3.8 | 59 | 18 |
| 19 | 23.8 | 3.7 | 27 | 8 |
| 20 | 24.3 | 3.6 | 112 | 34 |
| 21 | 25.1 | 3.5 | 22 | 7 |
| 22 | 25.6 | 3.4 | 11 | 3 |
| 23 | 26.3 | 3.3 | 140 | 43 |
| 24 | 26.7 | 3.3 | 326 | 100 |
| 25 | 27.1 | 3.2 | 123 | 38 |
| 26 | 27.5 | 3.2 | 40 | 12 |
| 27 | 27.8 | 3.1 | 39 | 12 |
| 28 | 28.1 | 3.1 | 15 | 5 |
| 29 | 29.0 | 3.0 | 43 | 13 |
| 30 | 29.4 | 3.0 | 18 | 6 |
| 31 | 30.7 | 2.9 | 15 | 5 |
| 32 | 31.0 | 2.8 | 12 | 4 |
| 33 | 32.2 | 2.7 | 40 | 12 |
| 34 | 32.5 | 2.7 | 43 | 13 |
| 35 | 33.0 | 2.7 | 27 | 8 |
| 36 | 33.5 | 2.6 | 15 | 5 |
| 37 | 33.9 | 2.6 | 18 | 6 |
| 38 | 34.3 | 2.6 | 21 | 6 |
| 39 | 34.9 | 2.5 | 13 | 4 |
| 40 | 36.1 | 2.4 | 19 | 6 |
| 41 | 36.4 | 2.4 | 18 | 6 |
| 42 | 36.8 | 2.4 | 11 | 3 |
| 43 | 39.1 | 2.2 | 25 | 8 |

TABLE 27
XRPD Peak List for Form C

| | Position | | | |
|---|---|---|---|---|
| Peak No. | (°2#) | d-spacing | I (intensity) | I/Io |
| 1 | 13.5 | 6.5 | 12 | 3 |
| 2 | 13.9 | 6.3 | 122 | 35 |
| 3 | 14.7 | 5.9 | 22 | 6 |
| 4 | 15.3 | 5.7 | 74 | 22 |
| 5 | 16.2 | 5.4 | 59 | 17 |
| 6 | 16.7 | 5.2 | 39 | 11 |
| 7 | 17.7 | 4.9 | 85 | 25 |
| 8 | 18.1 | 4.8 | 36 | 10 |
| 9 | 20.2 | 4.3 | 77 | 22 |
| 10 | 20.6 | 4.2 | 40 | 12 |
| 11 | 21.0 | 4.2 | 44 | 13 |
| 12 | 21.6 | 4.0 | 74 | 22 |
| 13 | 22.1 | 4.0 | 322 | 94 |
| 14 | 23.2 | 3.8 | 26 | 8 |
| 15 | 23.8 | 3.7 | 33 | 10 |
| 16 | 24.2 | 3.6 | 57 | 17 |
| 17 | 24.7 | 3.5 | 28 | 8 |
| 18 | 25.1 | 3.5 | 237 | 69 |
| 19 | 25.7 | 3.4 | 106 | 31 |
| 20 | 26.1 | 3.4 | 23 | 7 |
| 21 | 27.3 | 3.2 | 69 | 20 |
| 22 | 27.7 | 3.2 | 344 | 100 |
| 23 | 28.1 | 3.1 | 109 | 32 |
| 24 | 28.4 | 3.1 | 86 | 25 |
| 25 | 28.7 | 3.1 | 87 | 25 |
| 26 | 29.8 | 2.9 | 42 | 12 |
| 27 | 30.5 | 2.9 | 51 | 15 |
| 28 | 30.9 | 2.8 | 26 | 8 |
| 29 | 31.7 | 2.8 | 25 | 7 |
| 30 | 32.3 | 2.7 | 26 | 8 |
| 31 | 32.7 | 2.7 | 56 | 16 |
| 32 | 33.5 | 2.6 | 31 | 9 |
| 33 | 34.7 | 2.5 | 41 | 12 |
| 34 | 35.6 | 2.5 | 20 | 6 |
| 35 | 35.8 | 2.5 | 22 | 6 |
| 36 | 38.9 | 2.3 | 17 | 5 |
| 37 | 39.1 | 2.2 | 26 | 8 |
| 38 | 39.6 | 2.2 | 23 | 7 |

TABLE 28
XRPD Peak List for Form X.

| Peak No. | Position (°2#) | d-spacing | I (intensity) | I/Io |
|---|---|---|---|---|
| 1 | 5.4 | 16.3 | 16 | 10 |
| 2 | 6.2 | 14.2 | 6 | 4 |
| 3 | 8.2 | 10.6 | 6 | 4 |
| 4 | 9.5 | 9.2 | 9 | 5 |
| 5 | 10.2 | 8.6 | 23 | 14 |
| 6 | 10.9 | 8.0 | 6 | 4 |
| 7 | 11.2 | 7.8 | 7 | 4 |
| 8 | 12.4 | 7.1 | 12 | 7 |
| 9 | 15.5 | 5.6 | 29 | 18 |
| 10 | 16.2 | 5.4 | 164 | 100 |
| 11 | 17.5 | 5.0 | 40 | 24 |
| 12 | 18.6 | 4.7 | 11 | 7 |
| 13 | 19.5 | 4.5 | 13 | 8 |
| 14 | 20.3 | 4.3 | 39 | 24 |
| 15 | 21.4 | 4.1 | 11 | 7 |
| 16 | 21.9 | 4.0 | 14 | 9 |
| 17 | 22.8 | 3.8 | 30 | 18 |
| 18 | 23.2 | 3.8 | 58 | 35 |
| 19 | 24.4 | 3.6 | 40 | 24 |
| 20 | 24.8 | 3.5 | 39 | 24 |
| 21 | 25.7 | 3.4 | 29 | 18 |
| 22 | 26.4 | 3.3 | 18 | 11 |
| 23 | 27.0 | 3.2 | 31 | 19 |
| 24 | 27.7 | 3.2 | 15 | 9 |
| 25 | 28.4 | 3.1 | 22 | 13 |
| 26 | 29.7 | 3.0 | 14 | 9 |
| 27 | 30.1 | 2.9 | 10 | 6 |
| 28 | 30.9 | 2.8 | 5 | 3 |
| 29 | 32.9 | 2.7 | 12 | 7 |
| 30 | 33.4 | 2.6 | 9 | 5 |
| 31 | 33.9 | 2.6 | 11 | 7 |
| 32 | 34.6 | 2.5 | 6 | 4 |
| 33 | 35.6 | 2.5 | 7 | 4 |
| 34 | 36.1 | 2.4 | 5 | 3 |
| 35 | 38.2 | 2.3 | 5 | 3 |

The characterising peaks for Form A are at 8.3 and 26.8° 2#. Form A is also characterised by the absence of peaks in the 13.3 to 14.7° 2# region.

The most intense peaks for Form A are at 15.0, 16.2, 24.2 and 26.8° 2#. Less intense peaks are at 4.9, 12.9, 19.8, 21.8 and 22.9° 2#.

Form B is characterised by peaks at 8.0 and 8.6° 2#. Form B has a further unique peak at 14.4° 2#. Another peak at 13.6° 2# distinguishes Form B from Form A.

The most intense peaks for Form B are at 16, 24.3 and 26.7° 2#. Further, less intense peaks are at 4.8, 12.7, 15.2, 21.7 and 22.9° 2#.

Form C is characterised by peaks at 13.9 and 18.1° 2#. Other peaks which distinguish Form C from Form X are at 17.7, 22.1 and 23.2 and 24.7° 2#. Peaks which distinguish Form C from Form A are at 15.3 and 20.2° 2#. A peak which distinguishes Form C from Form B is at 16.7° 2#.

The most intense peaks for Form C are at 13.9, 22.1, 25.1, 25.7 and 27.7° 2#.

Further less intense peaks are at 16.2, 16.7, 18.1, 21.0 and 24.2° 2#.

Form X is characterised by peaks at 5.4, 10.2, 12.4 and 18.6° 2#. Further peaks are at 6.2, 9.5 and 11.2° 2#. An intense peak is at 16.2° 2#.

FT-IR Spectroscopy

Infrared spectra were acquired on a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. A Thunderdome accessory was used for sampling. A background data set was acquired with a clean Ge crystal. A Log 1/R(R=reflectance) spectrum was acquired by taking a ratio of these two data sets against each other. Wavelength calibration was performed using polystyrene. Further parameters are as follows.

TABLE 29
FT-IR Parameters

| | Form A | Form C |
|---|---|---|
| Number of sample scans | 128 | 256 |
| Number of background | 128 | 256 |
| scans | | |
| Resolution/cm⁻¹ | 4.000 | 4.000 |
| Sample gain | 2.0 | 2.0 |
| Mirror velocity | 0.6329 | 0.6329 |
| Aperture | 100.00 | 100.00 |
| Detector | DTGS KBr | DTGS KBr |
| Beamsplitter | XT-KBr | XT-KBr |

The FT-IR spectra differentiate Form A from Form C.

Differential Scanning Calorimetry (DSC) & Thermogravimetry (TG)

The DSC analysis was carried out on a TA Instruments differential scanning calorimeter 2920. The instrument was calibrated using indium as the reference material. The sample was placed into a standard aluminum DSC pan, the pan was crimped, and the weight accurately recorded. The sample was equilibrated at 25° C. and heated under a nitrogen purge at a rate of 10° C./min up to 350° C. Indium metal was used as calibration standard.

Thermogravimetric (TG) analyses were performed using a TA Instruments 2950 thermogravimetric analyzer. Each sample was placed in an aluminum sample pan and inserted into the TG furnace. The furnace was heated under nitrogen at a rate of 10° C./min, up to a final temperature of 350° C. Nickel and Alumel were used as the calibration standards.

The DSC thermogram of Form A exhibited an endotherm at approximately 210 (206-213) and two minor endotherms at 220, and 260° C., followed by an exotherm at approximately 295° C. (FIG. 9). Based on hot stage data (not shown), the first two endotherms were identified as melting endotherms, and the third endotherm and the exotherm corresponded to decomposition.

Thermal data for Form B (FIG. 10) appeared very similar to thermal data for Form A (FIG. 9). The initial broad endotherm at approximately 67° C. likely corresponded to loss of water. As the other three endotherms in DSC occurred in the same temperature range, Form B most likely converted to Form A on drying at elevated temperatures.

Thermal data for Form C are presented in FIG. 11. The TG data showed an insignificant weight loss of approximately 0.4% from 25 to 220° C. suggesting that the material was not solvated or hydrated. The baseline in DSC between 25 and 220° C. indicated that the weight loss in TG might be due to loss of residual solvent (water). The DSC thermogram exhibited a sharp endotherm at approximately 241° C. followed by decomposition. Hotstage data confirmed that the endotherm was due to the melt.

Coulometric Karl Fischer Titration

Coulometric Karl Fischer (KF) analysis for water determination was performed using a Mettler Toledo DL39 Karl Fischer titrator. Samples were placed in the KF titration vessel containing approximately Hydranal—Coulomat AD and mixed for 60 seconds to ensure dissolution. The sample was then titrated by means of a generator electrode which produces iodine by electrochemical oxidation: 2I−=>I2+2e. Three replicates were obtained to ensure reproducibility. A NIST-traceable water standard (Hydranal Water Standard 10.0) was analyzed to check the operation of the coulometer. Data were collected and analyzed using LabX Pro Titration v2.10.000.

An initial batch of Form A contained approximately 2.75% or 0.6 moles of water.

After it had been vacuum-dried at approximately 70° C. for 1 week, the water content was reduced to approximately 0.44% (0.09 mole). The XRPD pattern of the dry material was very similar to that of the starting Form A, except for the peak shifts observed at lower degrees 2 Theta and between approximately 20 and 24 degrees 2 Theta.

Form A was found to be stable at 31% relative humidity after two weeks, and at 43% relative humidity after five days.

Form A samples were stored at lower relative humidities of approximately 9-11, 23 and 32% RH and analyzed by XRPD and Karl-Fischer titration analysis after four weeks (Tables 30 and 31). The samples appeared to be Form A by XRPD. The water content was found to be approximately 1.7% (0.33 mole), 2.96% (0.59 mole), and 3.24% (0.65 mole), respectively. Note that the water content in the samples at 23 and 32% RH was slightly higher compared to the initial batch of Form A.

TABLE 30
Karl-Fischer Data for compound 1, Form A

| % RH | Time | Observations | % water | Moles of water |
|---|---|---|---|---|
| ~9-11 | 11 days | — | 1.70 | 0.33 |
| ~23 | 4 weeks | looks dry | 2.96 | 0.59 |
| ~32 | 4 weeks | looks dry | 3.24 | 0.65 |

TABLE 31
RH Stability of compound 1, Form A

| % RH | Time | XRPD Result |
|---|---|---|
| ~9-11 | 36 days | A |
| ~23 | 4 weeks | A |
| ~32 | 4 weeks | A |

After two days, the water content in Form B samples at 75 and 90% RH was 6.2% (1.28 mole) and 6.7% (1.39 mole), respectively (Table 32). As the starting Form A contained approximately 2.75% (0.6 mole) of water, less than one mole of water was gained in the relative humidity jars at both relative humidities (Table 33).

Form B samples were stable based on the observation that the XPRD patterns did not change after 2 days, 3, 7, and 8 weeks at both relative humidities. No significant changes in the water content were observed for the samples after 2 days, 3 weeks, and 8 weeks at both 75 and 90% RH.

TABLE 32
Karl-Fischer Data for compound 1, Form B

| % RH | Time | Observations | % water | Moles of water |
|---|---|---|---|---|
| ~55 | 8 days | looks dry | 5.34 | 1.1 |
| ~75 | 2 days | — | 6.23 | 1.28 |
| | 3 weeks | looks dry | 6.02 | 1.24 |
| | 8 weeks | looks dry | 6.12 | 1.26 |
| ~90 | 2 days | — | 6.70 | 1.39 |
| | 3 weeks | looks dry | 5.99 | 1.23 |
| | 8 weeks | looks dry | 6.46 | 1.33 |

TABLE 33
RH Stability of compound 1, Form B

| | | | Weight | Weight | XRPD |
|---|---|---|---|---|---|
| % RH | Time | Observations | change, mg | change, % | Result |
| ~55 | 8 days | looks dry | 10.7 | 9.5 | B |
| ~75 | 2 days | — | 11.0 | 4.5 | B |
| | 3 weeks | looks dry | — | — | B |
| | 7 weeks | looks dry | | | B |
| | 8 weeks | looks dry | | | B |
| ~90 | 2 days | — | 11.6 | 4.6 | B |
| | 3 weeks | looks dry | — | — | B |
| | 7 weeks | looks dry | | | B |
| | 8 weeks | looks dry | | | B |

Karl-Fischer data for Form C showed that the water content for a range of Form C samples varied from 0.04 moles to 0.07 moles. This result further indicates that Form C is anhydrous, the water present being residual water.

Form X did not change by XRPD after 6 days and 2 weeks of drying at approximately 70° C. Based on the proton NMR the resulting material contained no methanol. The water content in the dried samples was reduced to approximately 2.72% (0.55 mole) and 1.31% (0.26 mole), respectively.

Form X converted to Form B after 4 days and 5 weeks at 90% RH. As mentioned above, Form B can be dehydrated upon desorption (drying) to convert back to Form A. Thus, if Form X is generated in the crystallisation process as a side-product it can be converted to the desired Form A by subjecting it to high relative humidities with subsequent drying.

Form X samples were stored at relative humidities of approximately 43, 75 and 90% RH and analyzed by XRPD and Karl-Fischer titration analysis after five weeks (Tables 34 and 35).

TABLE 34
Karl-Fischer Data for Low Crystalline Form X

| | | | | Moles of | Resulting |
|---|---|---|---|---|---|
| % RH | Time | Observations | % water | water | Form |
| 43 | 5 weeks | looks dry | 5.52 | 1.13 | X |
| 75 | 5 weeks | looks dry | 8.74 | 1.85 | X |
| 90 | 5 weeks | looks dry | 6.47 | 1.34 | B |

TABLE 35
RH Stability of compound 1, Low Crystalline Form X

| | | | Weight | Weight | XRPD |
|---|---|---|---|---|---|
| % RH | Time | Observations | change, mg | change, % | Result |
| ~43 | 7 days | looks dry | 1.2 | 3.4 | X |
| | 5 weeks | looks dry | — | — | X |
| ~75 | 7 days | looks dry | 2.4 | 6.0 | X |
| | 5 weeks | looks dry | — | — | X |
| ~90 | 1 day | — | 2.3 | 5.6 | X |
| | 4 days | — | — | — | B |
| | 7 days | looks dry | 0.9 | 2.1 | — |
| | 5 weeks | looks dry | — | — | B |

For the preparation of pharmaceutical compositions of the polymorphs of (R)-5-(2-aminoethyl)-1-(6,8-difluorochroman-3-yl)-1,3-dihydroimidazole-2-thione hydrochloride, inert pharmaceutically acceptable carriers are admixed with the active compound. The pharmaceutically acceptable carriers may be either solid or liquid. Solid form preparations include powders, tablets, dispersible granules and capsules. A solid carrier can be one or more substances which may also act as diluents, flavouring agents, solubilizers, lubricants, suspending agents, binders or tablet disintegrating agents; it may also be an encapsulating material.

Preferably the pharmaceutical preparation is in unit dosage form, e.g. packaged preparation, the package containing discrete quantities of preparation such as packeted tablets, capsules and powders in vials or ampoules.

The dosages may be varied depending on the requirement of the patient, the severity of the disease and the particular compound being employed. For convenience, the total daily dosage may be divided and administered in portions throughout the day. It is expected that once or twice per day administration will be most suitable. Determination of the proper dosage for a particular situation is within the skill of those in the medical art.

It will be appreciated that the invention may be modified within the scope of the appended claims.

1. Crystalline Form A of (R)-5-(2-Amino ethyl)-1-(6,8-difluoro chroman-3-yl)-1,3-dihydroimidazole-2-thione hydrochloride having an XRPD pattern with peaks at 8.3 and 26.8 0.2° 2#.

2. Crystalline Form A of (R)-5-(2-Amino ethyl)-1-(6,8-difluoro chroman-3-yl)-1,3-dihydroimidazole-2-thione hydrochloride according to claim 1, having an XRPD pattern with further peaks at 15.0, 16.2, and 24.2±0.2° 2#.

3. Crystalline Form A of (R)-5-(2-Aminoethyl)-1-(6,8-difluorochroman-3-yl)-1,3-dihydroimidazole-2-thione hydrochloride according to claim 1 or 2, having an XRPD pattern with further peaks at 4.9, 12.9, 19.8, 21.8 and 22.9±0.2° 2#.

4. Crystalline Form A of (R)-5-(2-Aminoethyl)-1-(6,8-difluorochroman-3-yl)-1,3-dihydroimidazole-2-thione hydrochloride having the XRPD pattern of FIG. 1.

5. Crystalline Form A of (R)-5-(2-Aminoethyl)-1-(6,8-difluorochroman-3-yl)-1,3-dihydroimidazole-2-thione hydrochloride according to any preceding claim, wherein Form A is a variable hydrate with the number of moles of water being dependent on the relative humidity and varying from about 0.09 to about 0.65 moles.

6. Crystalline Form A of (R)-5-(2-Aminoethyl)-1-(6,8-difluorochroman-3-yl)-1,3-dihydroimidazole-2-thione hydrochloride having characteristic FT-IR peaks at 1491.90, 1220.70, 1117.50, 1039.50, 851.80 and 747.00 cm⁻¹.

7. Crystalline Form A of (R)-5-(2-Aminoethyl)-1-(6,8-difluorochroman-3-yl)-1,3-dihydroimidazole-2-thione hydrochloride according to claim 6, further having characteristic FT-IR peaks at 3053.30, 1599.80, 1406.10, 1330.70, 1287.60, 1194.00, 985.50 and 713.70 cm⁻¹.

8. Crystalline Form A of (R)-5-(2-Aminoethyl)-1-(6,8-difluorochroman-3-yl)-1,3-dihydroimidazole-2-thione hydrochloride according to claim 6 or 7, further having characteristic FT-IR peaks at 2939.70, 1448.30 and 1244.50 cm⁻¹.

9. Crystalline Form A of (R)-5-(2-Aminoethyl)-1-(6,8-difluorochroman-3-yl)-1,3-dihydroimidazole-2-thione hydrochloride having the FT-IR spectrum of FIG. 6.

10. Crystalline Form A of (R)-5-(2-Aminoethyl)-1-(6,8-difluorochroman-3-yl)-1,3-dihydroimidazole-2-thione hydrochloride having the DSC spectrum of FIG. 9.

11. Crystalline Form A of (R)-5-(2-Amino ethyl)-1-(6,8-difluorochroman-3-yl)-1,3-dihydroimidazole-2-thione hydrochloride having a purity greater than or equal to 99.0%.

12. Crystalline Form A of (R)-5-(2-Aminoethyl)-1-(6,8-difluorochroman-3-yl)-1,3-dihydroimidazole-2-thione hydrochloride having a purity in the range of 99.0% to 99.8%.

13. Crystalline Form A of (R)-5-(2-Amino ethyl)-1-(6,8-difluorochroman-3-yl)-1,3-dihydroimidazole-2-thione hydrochloride having a purity of 99.5%.

14. Crystalline Form B of (R)-5-(2-Aminoethyl)-1-(6,8-difluorochroman-3-yl)-1,3-dihydroimidazole-2-thione hydrochloride having an XRPD pattern with peaks at 8.0 and 8.6±0.2° 2#.

15. Crystalline Form B of (R)-5-(2-Aminoethyl)-1-(6,8-difluorochroman-3-yl)-1,3-dihydroimidazole-2-thione hydrochloride according to claim 14, having an XRPD pattern with further peaks at 13.6, 14.4, 16.0, 24.3 and 26.7±0.2° 2#.

16. Crystalline Form B of (R)-5-(2-Aminoethyl)-1-(6,8-difluorochroman-3-yl)-1,3-dihydroimidazole-2-thione hydrochloride according to claim 14 or 15, having an XRPD pattern with further peaks at 4.8, 12.7, 13.6, 14.4, 15.2, 21.7 and 22.9±0.2° 2#.

17. Crystalline Form B of (R)-5-(2-Aminoethyl)-1-(6,8-difluorochroman-3-yl)-1,3-dihydroimidazole-2-thione hydrochloride having the XRPD pattern of FIG. 2.

18. Crystalline Form B of (R)-5-(2-Aminoethyl)-1-(6,8-difluorochroman-3-yl)-1,3-dihydroimidazole-2-thione hydrochloride according to any one of claims 14 to 17, wherein Form B is a variable hydrate with the number of moles of water being dependent on the relative humidity and varying from about 1.1 to about 1.4 moles.

19. Crystalline Form B of (R)-5-(2-Aminoethyl)-1-(6,8-difluorochroman-3-yl)-1,3-dihydroimidazole-2-thione hydrochloride according to any one of claims 14 to 18, wherein Form B is a monohydrate.

20. Crystalline Form B of (R)-5-(2-Aminoethyl)-1-(6,8-difluorochroman-3-yl)-1,3-dihydroimidazole-2-thione hydrochloride having the DSC spectrum of FIG. 10.

21. Crystalline Form B of (R)-5-(2-Aminoethyl)-1-(6,8-difluorochroman-3-yl)-1,3-dihydroimidazole-2-thione hydrochloride having a purity greater than or equal to 99.0%.

22. Crystalline Form B of (R)-5-(2-Aminoethyl)-1-(6,8-difluorochroman-3-yl)-1,3-dihydroimidazole-2-thione hydrochloride having a purity in the range of 99.0% to 99.8%.

23. Crystalline Form B of (R)-5-(2-Amino ethyl)-1-(6,8-difluorochroman-3-yl)-1,3-dihydroimidazole-2-thione hydrochloride having a purity of 99.5%.

24. Crystalline Form C of (R)-5-(2-Aminoethyl)-1-(6,8-difluorochroman-3-yl)-1,3-dihydroimidazole-2-thione hydrochloride having an XRPD pattern with peaks at 13.9, 18.1, 22.1, 25.1 and 25.7±0.2° 2#.

25. Crystalline Form C of (R)-5-(2-Aminoethyl)-1-(6,8-difluorochroman-3-yl)-1,3-dihydroimidazole-2-thione hydrochloride according to claim 24, having an XRPD pattern with further peaks at 15.3, 17.7 and 20.2±0.2° 2#.

26. Crystalline Form C of (R)-5-(2-Aminoethyl)-1-(6,8-difluorochroman-3-yl)-1,3-dihydroimidazole-2-thione hydrochloride according to claim 24 or 25, having an XRPD pattern with further peaks at 16.2, 16.7, 21.0 and 24.2±0.2° 2#.

27. Crystalline Form C of (R)-5-(2-Aminoethyl)-1-(6,8-difluorochroman-3-yl)-1,3-dihydroimidazole-2-thione hydrochloride having the XRPD pattern of FIG. 3.

28. Crystalline Form C of (R)-5-(2-Aminoethyl)-1-(6,8-difluorochroman-3-yl)-1,3-dihydroimidazole-2-thione hydrochloride having characteristic FT-IR peaks at 1492, 1220.2, 1117.4, 1033.4, 845.2, 792.6 and 750.1 cm⁻¹.

29. Crystalline Form C of (R)-5-(2-Aminoethyl)-1-(6,8-difluorochroman-3-yl)-1,3-dihydroimidazole-2-thione hydrochloride according to claim 28, further having characteristic FT-IR peaks at 3041.70, 1596.50, 1403.40, 1333.80, 1290.90, 1173.20, 1078.10, 984.90 and 713.20 cm⁻¹.

30. Crystalline Form C of (R)-5-(2-Aminoethyl)-1-(6,8-difluorochroman-3-yl)-1,3-dihydroimidazole-2-thione hydrochloride having the FT-IR spectrum of FIG. 7.

31. Crystalline Form C of (R)-5-(2-Aminoethyl)-1-(6,8-difluorochroman-3-yl)-1,3-dihydroimidazole-2-thione hydrochloride having a purity greater than or equal to 99.0%.

32. Crystalline Form C of (R)-5-(2-Aminoethyl)-1-(6,8-difluorochroman-3-yl)-1,3-dihydroimidazole-2-thione hydrochloride having a purity in the range of 99.0% to 99.8%.

33. Crystalline Form C of (R)-5-(2-Aminoethyl)-1-(6,8-difluorochroman-3-yl)-1,3-dihydroimidazole-2-thione hydrochloride having a purity of 99.5%.

34. Crystalline Form X of (R)-5-(2-Amino ethyl)-1-(6,8-difluorochroman-3-yl)-1,3-dihydroimidazole-2-thione hydrochloride having an XRPD pattern with peaks at 5.4, 10.2, 12.4 and 18.6±° 2#.

35. Crystalline Form X of (R)-5-(2-Aminoethyl)-1-(6,8-difluoro chroman-3-yl)-1,3-dihydroimidazole-2-thione hydrochloride according to claim 34, having an XRPD pattern with further peaks at 6.2, 9.5, 11.2 and 16.2±° 2#.

36. Crystalline Form X of (R)-5-(2-Amino ethyl)-1-(6,8-difluoro chroman-3-yl)-1,3-dihydroimidazole-2-thione hydrochloride having the XRPD pattern of FIG. 4.

37. Crystalline Form X of (R)-5-(2-Aminoethyl)-1-(6,8-difluorochroman-3-yl)-1,3-dihydroimidazole-2-thione hydrochloride having a purity greater than or equal to 99.0%.

38. Crystalline Form X of (R)-5-(2-Amino ethyl)-1-(6,8-difluorochroman-3-yl)-1,3-dihydroimidazole-2-thione hydrochloride having a purity in the range of 99.0% to 99.8%.

39. Crystalline Form X of (R)-5-(2-Amino ethyl)-1-(6,8-difluoro chroman-3-yl)-1,3-dihydroimidazole-2-thione hydrochloride having a purity of 99.5%.

40. A process for preparing crystalline Form A of (R)-5-(2-Aminoethyl)-1-(6,8-difluorochroman-3-yl)-1,3-dihydroimidazole-2-thione hydrochloride comprising recrystallising (R)-5-(2-Aminoethyl)-1-(6,8-difluorochroman-3-yl)-1,3-dihydroimidazole-2-thione ydrochloride in aqueous HCl.

41. A process according to claim 41, wherein the recrystallisation comprises (a) dissolving (R)-5-(2-Aminoethyl)-1-(6,8-difluorochroman-3-yl)-1,3-dihydroimidazole-2-thione hydrochloride in aqueous HCl, (b) filtering the solution, (c) cooling the solution with stirring, and (d) isolating, washing and drying the precipitated Form A.

42. A process for preparing crystalline Form A of (R)-5-(2-Aminoethyl)-1-(6,8-difluorochroman-3-yl)-1,3-dihydroimidazole-2-thione hydrochloride comprising forming (R)-5-(2-Aminoethyl)-1-(6,8-difluorochroman-3-yl)-1,3-dihydroimidazole-2-thione hydrochloride in situ and crystallising Form A using aqueous HCl.

43. A process according to claim 42, wherein the crystallisation comprises (a) adding aqueous HCl to a solution of (R)-5-(2-Aminoethyl)-1-(6,8-difluorochroman-3-yl)-1,3-dihydroimidazole-2-thione hydrochloride, (b) cooling the solution with stirring and (c) isolating, washing and drying the precipitated Form A.

44. A process for preparing crystalline Form B of (R)-5-(2-Aminoethyl)-1-(6,8-difluorochroman-3-yl)-1,3-dihydroimidazole-2-thione hydrochloride comprising subjecting Form A of (R)-5-(2-Amino ethyl)-1-(6,8-difluoro chroman-3-yl)-1,3-dihydroimidazole-2-thione hydrochloride to 43% to 90% relative humidity.

45. A process according to claim 44, wherein the relative humidity is from 55% to 65%.

46. A process according to claim 44 or 45, wherein the subjecting step takes place within a time range from 1 day to 2 weeks.

47. A process according to claim 44 or 45, wherein the subjecting step takes place over 1 to 2 days.

48. A process according to claim 44, 45, 46 or 47, wherein the subjecting step takes place at 25° C.

49. A process for preparing crystalline Form B of (R)-5-(2-Aminoethyl)-1-(6,8-difluorochroman-3-yl)-1,3-dihydroimidazole-2-thione hydrochloride comprising dissolving or slurrying Form A of (R)-5-(2-Aminoethyl)-1-(6,8-difluorochroman-3-yl)-1,3-dihydroimidazole-2-thione hydrochloride in an organic solvent, or mixtures of organic solvents, filtering the solution and allowing the solvent to evaporate.

50. A process according to claim 49, wherein the organic solvent is selected from ethyl ether, hexane, acetonitrile, 1,4-dioxane, ethanol, ethyl acetate, hexafluoroisopropanol, methanol, methylene chloride, methyl ethyl ketone, toluene, propionitrile, trifluorotoluene, cyclohexane, methyl iso-butyl ketone, n-butyl acetate, acetone, toluene, iso-propyl ether and mixtures thereof.

51. A process according to claim 49 or 50, wherein the solvent is allowed to evaporate from an open vial.

52. A process according to claim 49 or 50, wherein the solvent is allowed to evaporate from a vial covered with a perforated material.

53. A process for preparing crystalline Form C of (R)-5-(2-Aminoethyl)-1-(6,8-difluorochroman-3-yl)-1,3-dihydroimidazole-2-thione hydrochloride comprising subjecting Form A or B of (R)-5-(2-Amino ethyl)-1-(6,8-difluorochroman-3-yl)-1,3-dihydroimidazole-2-thione hydrochloride in a solution of ethanol or ethanol/solvent mixtures to evaporation under nitrogen.

54. A process for preparing crystalline Form C of (R)-5-(2-Aminoethyl)-1-(6,8-difluorochroman-3-yl)-1,3-dihydroimidazole-2-thione hydrochloride comprising: (a) stirring a mixture of (R)-5-(2-Aminoethyl)-1-(6,8-difluorochroman-3-yl)-1,3-dihydroimidazole-2-thione hydrochloride in a first organic solvent and an aqueous solution of a base, wherein the first organic solvent is immiscible with water; (b) extracting the organic phase and evaporating the product to dryness; (c) dissolving the product of (b) in dry ethanol; (d) acidifying the product of step (c) with HCl in ethanol; (e) collecting the precipitate; (f) washing the precipitate with ethanol; and (g) drying the product of step (f) to yield Form C.

55. A process according to claim 54, wherein the first organic solvent is ethyl acetate.

56. A process according to claim 54 or 55, wherein the precipitate is collected hot.

57. A process according to any of claims 54 to 56, wherein the (R)-5-(2-Aminoethyl)-1-(6,8-difluorochroman-3-yl)-1,3-dihydroimidazole-2-thione hydrochloride is prepared prior to step (a) and converted to Form C in situ by steps (a) to (g).

58. A process according to any of claims 54 to 56, wherein the (R)-5-(2-Aminoethyl)-1-(6,8-difluorochroman-3-yl)-1,3-dihydroimidazole-2-thione hydrochloride is prepared prior to step (a), isolated and then converted to Form C by steps (a) to (g).

59. A process for preparing crystalline Form C of (R)-5-(2-Amino ethyl)-1-(6,8-difluorochroman-3-yl)-1,3-dihydroimidazole-2-thione hydrochloride comprising slurrying Form A of (R)-5-(2-Aminoethyl)-1-(6,8-difluorochroman-3-yl)-1,3-dihydroimidazole-2-thione hydrochloride in acetonitrile and isolating Form C by vacuum filtration.

60. A process according to claim 59, wherein the slurrying is carried out for a period of from 4 days to 7 days.

61. A process for preparing crystalline Form C of (R)-5-(2-Aminoethyl)-1-(6,8-difluorochroman-3-yl)-1,3-dihydroimidazole-2-thione hydrochloride comprising preparing a saturated solution of Form A of (R)-5-(2-Aminoethyl)-1-(6,8-difluorochroman-3-yl)-1,3-dihydroimidazole-2-thione hydrochloride in methanol at an elevated temperature, filtering the warm solution, cooling the solution, and isolating the Form C.

62. A process according to claim 61, wherein the cooling brings the temperature of the solution to room temperature.

63. A process according to claim 61 or 62, wherein the solids are isolated by decantation followed by air drying.

64. A process for preparing crystalline Form X of (R)-5-(2-Aminoethyl)-1-(6,8-difluorochroman-3-yl)-1,3-dihydroimidazole-2-thione hydrochloride comprising dissolving Form A of (R)-5-(2-Aminoethyl)-1-(6,8-difluorochroman-3-yl)-1,3-dihydroimidazole-2-thione hydrochloride in methanol, filtering the solution and evaporating the methanol under a stream of nitrogen.

65. A process according to claim 64, wherein the evaporation is carried out at about 9% relative humidity.

66. A process according to claim 64 or 65, wherein the evaporation is carried out at room temperature.

67. A pharmaceutical formulation comprising Form A according to any of claims 1 to 13, Form B according to any of claims 14 to 23, Form C according to any of claims 24 to 33 or Form X according to any of claims 34 to 39 of (R)-5-(2-Aminoethyl)-1-(6,8-difluorochroman-3-yl)-1,3-dihydroimidazole-2-thione hydrochloride and one or more pharmaceutically acceptable carriers or excipients.

68. Form A according to any of claims 1 to 13, Foam B according to any of claims 14 to 23, Form C according to any of claims 24 to 33 or Form X according to any of claims 34 to 39 of (R)-5-(2-Aminoethyl)-1-(6,8-difluorochroman-3-yl)-1,3-dihydroimidazole-2-thione hydrochloride for use in medicine.

69. Use of Form A according to any of claims 1 to 13, Form B according to any of claims 14 to 23, Form C according to any of claims 24 to 33 or Form X according to any of claims 34 to 39 of (R)-5-(2-Aminoethyl)-1-(6,8-difluorochroman-3-yl)-1,3-dihydroimidazole-2-thione hydrochloride in the manufacture of a medicament for treatment of cardiovascular disorders.

70. Use of Form A according to any of claims 1 to 13, Form B according to any of claims 14 to 23, Faun C according to any of claims 24 to 33 or Form X according to any of claims 34 to 39 of (R)-5-(2-Aminoethyl)-1-(6,8-difluorochroman-3-yl)-1,3-dihydroimidazole-2-thione hydrochloride in the manufacture of a medicament for peripherally-selective inhibition of D#H.

71. A process for preparing the amorphous form of (R)-5-(2-Aminoethyl)-1-(6,8-difluorochroman-3-yl)-1,3-dihydroimidazole-2-thione hydrochloride comprising lyophilising an aqueous solution of Form A of (R)-5-(2-Aminoethyl)-1-(6,8-difluorochroman-3-yl)-1,3-dihydroimidazole-2-thione hydrochloride.

72. A process according to claim 71, wherein the lyophilisation takes place over a period of 2 to 5 days.

73. A process according to claim 72, wherein the lyophilisation takes place over a period of 3 to 4 days.
